(19) United States
(12) Patent Application Publication
(10) Pub. No.: US 2023/0181733 A1
(43) Pub. Date: Jun. 15, 2023

(54) COMPOSITIONS AND METHODS FOR STEM CELL TRANSPLANT CONDITIONING AND USES THEREOF

(71) Applicant: Jasper Therapeutics, Inc., Redwood City, CA (US)

(72) Inventors: Wendy Pang, Palo Alto, CA (US); Judith Shizuru, Palo Alto, CA (US); Robert Sikorski, Woodside, CA (US)

(21) Appl. No.: 18/047,604

(22) Filed: Oct. 18, 2022

Related U.S. Application Data
(60) Provisional application No. 63/257,008, filed on Oct. 18, 2021.
(60) Provisional application No. 63/314,923, filed on Feb. 28, 2022.
(60) Provisional application No. 63/334,602, filed on Apr. 25, 2022.

(51) Int. Cl. A61K 39/395; C12N 5/0789; C07K 16/28; A61K 45/06; A61K 35/28; A61K 31/7076; A61P 35/02; A61N 5/10

(52) U.S. Cl. CPC A61K 39/3955; A61K 31/7076; A61K 35/28; A61K 45/06; A61N 5/10; A61P 35/02; C07K 16/2803; C12N 5/0647; A61K 2039/545

(57) ABSTRACT

Provided herein are compositions and methods related to conditioning a subject for a hematopoietic cell transplant (HCT) using a combination of an inhibitor of a stem cell growth factor receptor (KIT), total body irradiation, and a chemotherapeutic agent. The compositions and methods described herein may be used to treat a subject in need of a transplant due to a variety of diseases or disorders, such as acute myeloid leukemia, myelodysplastic syndrome, and severe combined immune deficiency.

### CROSS-REFERENCE TO RELATED APPLICATIONS

This application claims priority to U.S. Provisional Pat. Application No. 63/257,008 filed Oct. 18, 2021, U.S. Provisional Pat. Application No. 63/314,923, filed Feb. 28, 2022, and U.S. Provisional Pat. Application No. 63/334,602 filed Apr. 25, 2022, the contents of which are incorporated by reference in their entirety.

### REFERENCE TO AN ELECTRONIC SEQUENCE LISTING

The contents of the electronic sequence listing (JATH_004_03US_SeqList_ST26.xml; Size: (41,168 bytes; and Date of Creation: Oct. 17, 2022) are herein incorporated by reference in its entirety.

### FIELD OF THE INVENTION

The present disclosure relates to compositions and methods for conditioning a subject for a hematopoietic cell transplant (HCT). The compositions and methods described herein may be used to treat patients requiring HCT for a variety of different diseases or disorders, including but not limited to myelodysplastic syndrome (MDS), acute myeloid leukemia (AML), and severe combined immune deficiency (SCID).

### BACKGROUND

Hematopoietic cell transplant (HCT) generally involves the intravenous infusion of autologous or allogeneic donor hematopoietic stem cells (HSC) and/or hematopoietic stem or progenitor cells (HSPCs) obtained from bone marrow, peripheral blood, or umbilical cord blood into a subject whose bone marrow or immune system is damaged or defective. HCT may be performed as part of therapy to treat a number of disorders, including cancers, such as leukemias, as well as congenital immunodeficiency disorders.

Myelodysplastic syndrome (MDS) and acute myeloid leukemia (AML) are hematologic malignancies primarily affecting older adults, while severe combined immunodeficiency (SCID) typically presents in infancy and results in profound immune deficiency. Allogeneic HCT is potentially curative for both MDS/AML and SCID patients. However, the toxicity associated with extant conditioning treatments limits its application.

HCT conditioning regimens clear or reduce bone-marrow niches of endogenous HSCs, thereby improving the success of donor HSC and/or HSPC engraftment. However, traditional conditioning regimens typically include treatment with total body irradiation (TBI) and/or chemotherapy, which, in dosages sufficient to substantially clear HSCs, exhibit toxic effects not tolerated by all patient populations. More recently, less toxic non-myeloblative treatments, such as anti-c-Kit antibodies, e.g., JSP191, have been developed.

Nonetheless, there remains a need for innovative conditioning regimens that exhibit low toxicity while still facilitating successful HCT engraftment. The present disclosure meets this need by providing conditioning regimens for HCT treatment of SCID and other disorders.

### BRIEF SUMMARY OF THE INVENTION

The present disclosure provides inter alia a method of conditioning a mammalian patient for a hematopoietic cell transplant (HCT), including administering to the patient an anti-c-Kit antibody; administering to the patient total body irradiation (TBI); and administering to the patient a chemotherapy, in a dose effective to deplete endogenous hematopoietic stem cells from the patient.

In one aspect, the disclosure provides a method of conditioning a mammalian subject for a hematopoietic cell transplant (HCT), the method comprising:
(a) administering to the subject an inhibitor of c-Kit, optionally an anti-c-Kit antibody;
(b) administering to the subject total body irradiation (TBI); and
(c) administering to the subject a chemotherapy,
in a dose effective to deplete endogenous hematopoietic stem cells from the subject.

In certain embodiments, the anti-c-Kit antibody comprises one or more complementarity-determining regions (CDRs) present in a monoclonal antibody selected from the group consisting of: SR-1, JSP191, MGTA-117, FSI-174, CDX-0159, CDX-0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112. In certain embodiments, the anti-c-Kit antibody comprises one or more complementarity-determining regions (CDRs) present in a humanized version of a monoclonal antibody selected from the group consisting of: ACK2, ACK4, 2B8, 3C11, MR-1, and CD122. In certain embodiments, the anti-c-Kit antibody comprises the CDRs of an antibody that blocks the binding of stem cell factor (SCF) to stem cell factor receptor (CD117), optionally wherein the antibody is JSP191. In certain embodiments, the subject is administered about 0.01 mg/kg to about 2 mg/kg of the anti-c-kit antibody, optionally wherein the subject is administered about 0.1 mg/kg to about 1 mg/kg of the anti-c-Kit antibody. In certain embodiments, the subject is administered TBI comprising about 50 cGy to about 5 Gy, optionally wherein the subject is administered TBI comprising about 1 Gy to about 3 Gy. In certain embodiments, the subject is administered about 10-50 mg/m²/day of the chemotherapy, optionally wherein the chemotherapy is selected from the group consisting of fludarabine and clofarabine, and optionally wherein the chemotherapy is administered for about one to about six days. In certain embodiments, the subject is administered about 0.6 mg/kg of the anti-c-Kit antibody, about 2 Gy of the TBI, and about 30 mg/m²/day of the chemotherapy before the HCT, optionally wherein the anti-c-Kit antibody is JSP191, and optionally wherein the chemotherapy is flutarabine. In certain embodiments, the anti-c-Kit antibody is administered to the subject intravenously and/or the chemotherapy is administered to the subject intravenously. In certain embodiments, the anti-c-Kit antibody is administered to the subject between about 5 to about 20 days prior to the HCT, optionally between about 10 to about 14 days prior to the HCT. In certain embodiments, the level of anti-c-Kit antibody in the subject determines the day of HCT for the subject, optionally wherein the day of transplant is within about 4 to about 10 days from the day the anti-c-Kit antibody is at a concentration of about 2000 ng/ml or less in a subject. In certain embodiments, the chemotherapy is administered to the subject between about one to about seven days prior to the HCT, optionally between about two to about four days prior to the HCT, optionally about three days prior to the HCT, and optionally wherein the chemotherapy is administered for about three days. In certain embodiments, the TBI is administered to the subject about zero to about three days prior to the HCT, optionally on the same day as the HCT. In certain embodiments, the subject is also administered one or more of:
(a) a graft versus host disease (GVHD) prophylactic agent, optionally selected from the group consisting of glucocorticoids, calcineurin inhibitor, tacromilus, sirolimus, methotrexate, mycophenolate mofetil, mycophenolic acid, cyclosporine A, rapamycin, FK506, corticosteroids, and CD40/CD40L inhibitors;
(b) ursodiol; and/or
(c) one or more of antibiotic, antifungal, and antiviral therapies.

In certain embodiments, the subject is a human sixty years or older. In certain embodiments, the subject has a hematopoietic cell transplantation comorbidity index (HCT-CI) greater than or equal to 3. In certain embodiments, the subject is in need of a HCT due to a disease or disorder selected from the group consisting of: a cancer, a cardiac disorder, a neural disorder, an autoimmune disease, an immunodeficiency, a metabolic disorder, a bone marrow failure disorder, and a genetic disorder. In certain embodiments, the cancer is a solid tissue cancer or a blood cancer, optionally a leukemia, a lymphoma, or a myelodysplastic syndrome (MDS). In certain embodiments, the cancer is multiple myeloma, acute lymphocytic leukemia (ALL), chronic lymphocytic leukemia (CLL), chronic myelogenous leukemia (CML), myelodysplastic syndromes (MDS), a myeloproliferative neoplasm, or acute myeloid leukemia (AML). In some embodiments, AML arises de novo in a subject. In some embodiments, AML arises from MDS. In certain embodiments, the immunodeficiency is a primary immune deficiency disease (PIDD), optionally severe combined immunodeficiency (SCID), combined immune deficiency (CID), leaky SCID, chronic granulomatous disease (CGD), or common variable immune deficiency (CVID). In certain embodiments, the bone marrow failure disorder is Fanconi anemia (FA), dyskeratosis congenita (DC), Shwachman-Diamond syndrome (SDS), congenital amegakaryocytic thrombocytopenia (CAMT), Blackfan-Diamond anemia (BDA), or reticular dysgenesis (RD).

In a related aspect, the disclosure provides a method of hematopoietic cell transplant (HCT) in a mammalian subject, the method comprising:
(a) conditioning a mammalian subj ect prior to and/or during and/or following the HCT according to the method disclosed herein; and
(b) transplanting hematopoietic stem cells (HSCs) and/or hematopoietic stem and pluripotent cells (HSPCs) into the subject, optionally wherein step (a) or step (b) are performed as outpatient procedures.

In certain embodiments, the HSCs and/or HSPCs are selected for CD34⁺ expression, optionally wherein the HSCs and/or HSPCs are purified, CD34⁺ Thy-1⁺ peripheral blood HSCs. In certain embodiments, the subject is transplanted with from 10⁵ to 10⁸ CD34⁺ HSCs and/or HSPCs /kg of the subject’s body weight. In certain embodiments, the HSCs and/or HSPCs are autologous or allogeneic to the subject, optionally wherein the autologous HSCs and/or HSPCs are gene-corrected. In certain embodiments, the HSCs and/or HSPCs are derived from bone marrow, cord blood, or peripheral blood of a donor. In certain embodiments, the subject is haploidentical relative to the HSCs and/or HSPCs. In certain embodiments, the HSCs and/or HSPCs are MHC matched to the subject. In certain embodiments, the method provides for at least 50%, 60%, 70%, 80%, 90%, or 95% donor CD15 myeloid cell chimerism following the HCT. In certain embodiments, minimal residual disease (MRD) and/or measurable residual disease (MRD) is undetected or reduced in the subject after a period of 28 days following the HCT. In certain embodiments, MID and/or MRD are detected by cytogenetics, flow cytometry, and/or next-generation sequencing (NGS).

In some embodiments, minimal residual disease (MRD) and/or measurable residual disease (MRD) is undetected or reduced in the subject following the HCT. In some embodiments, MRD is undetected or reduced after a period of 360 days following the HCT.

In some embodiments, severe chronic graft versus host disease (cGVHD) is undetected or reduced in the subject following HCT. In some embodiments, severe cGVHD is undetected or reduced after a period of 360 days following the HCT.

### BRIEF DESCRIPTION OF THE DRAWINGS

FIGS. 1A-1B provide schematic diagrams showing the difference between current myeloablative conditioning regimens (FIG. 1A) and conditioning via JSP191 (FIG. 1B).

FIG. 2 is a diagram showing how JSP191 binds to CD117 on HSCs and depletes HSCs.

FIGS. 3A and 3B are flow charts of dose finding cohorts for Severe Combined Immune Deficiency (SCID) patients in re-transplant and first transplant infant clinical trials of Example 1. FIG. 3A is a flow chart of dose finding cohorts for the re-transplant clinical trial. FIG. 3B is a flow chart of dose finding cohorts for the first transplant infant studies.

FIG. 4 is a table of subject demographics and summarized results of the first transplant infant study in Example 1.

FIG. 5 is graphs of T cells and NK cells in an example infant subject (0011, IL2RG, NK-) in response to hematopoietic cell transplant (HCT) conditioning with JSP191 alone.

FIG. 6 is a graph of the maximum proliferation of T cells as a percentage of CD3+ cells in response to phorbol myristate acetate (PHA) in the example infant subject of FIG. 5 (0011, IL2RG, NK-) with JSP191 HCT conditioning.

FIG. 7 is a table of the percentage of flow-sorted CD15+ donor myeloid chimerism in the example infant subject of FIGS. 5 and 6 (0011, IL2RG, NK-), with JSP191 HCT conditioning, before and after engraftment.

FIG. 8 is graphs of T cells and NK cells in a second example infant subject (0015, RAG2, NK+), in response to HCT conditioning with JSP191 alone.

FIG. 9 is a graph of JSP191 clearance in newly diagnosed (cohort B2) and re-transplanted SCID subjects (cohort A2).

FIG. 10 is a graph indicating re-transplant subjects’ response to hematopoietic cell transplantation.

FIG. 11 is a timeline of an illustrative conditioning regimen according to the disclosure, which was used in Example 2. Serial serum concentrations of JSP191 were obtained to predict the day subject JSP191 concentration was less than or equal to 2000 ng/mL. The day the subject exhibited this serum concentration established the day of transplant, i.e., this concentration was -4 days prior to transplant.

FIG. 12 is a table summarizing characteristics of the patients treated according to the Phase 1 trial described in Example 2.

FIGS. 13A and 13B provide results of the Phase 1 clinical trial, including JSP191 PK values (FIG. 13A) and engraftment (FIG. 13B) for the treated patients.

FIG. 14 is a table showing measurable residual disease (MRD) for the indicated patients at various timepoints before and following treatment.

FIGS. 15A-15D are graphs showing chimerism in patients at the indicated times following hematopoietic cell transplant (HCT). FIG. 15A shows total blood chimerism; FIG. 15B shows myeloid chimerism; FIG. 15C shows T cell chimerism; and FIG. 15D shows NK cell chimerism.

FIG. 16 is a table summarizing donor chimerism results for the indicated cell surface markers in various patients at the indicated times following HCT. CD15 is a myeloid cell marker; CD3 is a T cell marker; and CD56 is a natural killer (NK) cell marker.

FIG. 17 is a timeline of an example conditioning regimen following additional subject enrollment. Pharmacokinetic measurements and modeling of JSP191 were used to determine the Fludarabine (Flu) start date. Total body irradiation (TBI) was increased to 300 cGy to aid in lymphoablation. The mobilized peripheral blood (PB) HCT is shown as TD0.

FIG. 18 is a table showing the characteristics of all MDS and AML patients in the study following additional enrollment.

FIG. 19 is a graph of the pharmacokinetics of 0.6 mg/kg JSP191 in subjects demonstrating consistent and predictable clearance after administration.

FIG. 20 is a bar graph showing the percent depletion of CD34+CD45RA-CD117+ HSPC in the bone marrow of individual MDS and AML subjects with JSP191 administration. The average HSPC depletion was 67.5% (+/- 26.2%). The bars from left to right correspond to subjects having the following disease: AML from MDS; de novo AML; AML from MDS; MDS; MDS; de novo AML; MDS; de novo AML; MDS; de novo AML; MDS, de novo AML; and de novo AML.

FIG. 21 is a line plot of individual subject absolute neutrophil counts (ANC) showing depletion with JSP191, Flu, and TBI, and subsequent recovery of neutrophil levels after HCT (TD0).

FIGS. 22A-22D are line plots showing chimerism after HCT with subjects who had received a conditioning regimen including JSP191, Flu, and either 200 cGy TBI or 300 cGy TBI. FIG. 22A shows total chimerism. FIG. 22B shows myeloid chimerism. FIG. 22C shows T cell chimerism. FIG. 22D is a table of median donor chimerism, demonstrating that the total median donor chimerism was higher for conditioning regimens that included 300 cGy (TBI), 95% versus 200 cGy (91%).

FIGS. 23A-23B are diagrams showing the Measurable Residual Disease (MRD) status of AML patients (FIG. 23A) or MDS patients (FIG. 23B) over time TD28-TD360. QNS indicates “quantity not sufficient.” Subjects received a conditioning regimen including JSP191, Flu, and either 200 cGy TBI or 300 cGy TBI.

FIGS. 24A and 24B are line plots showing the Overall Survival (OS) (FIG. 24A) and Relapse and Treatment Related Mortality (TRM) (FIG. 24B) relative to the number of months post-hematopoietic cell transplant (HCT). Subjects received a conditioning regimen including JSP191, Flu, and either 200 cGy TBI or 300 cGy TBI.

FIG. 25 is a diagram showing the follow up Measurable Residual Disease (MRD) status of the AML patients shown in FIG. 23A. Subjects received a conditioning regimen including JSP191, Flu, and either 200 cGy TBI or 300 cGy TBI.

FIG. 26 is a table of outpatient hematopoietic cell transplant (HCT) patients who were hospitalized within the first 100 days. Subjects received a conditioning regimen including JSP191, Flu, and either 200 cGy TBI or 300 cGy TBI.

### DETAILED DESCRIPTION OF THE INVENTION

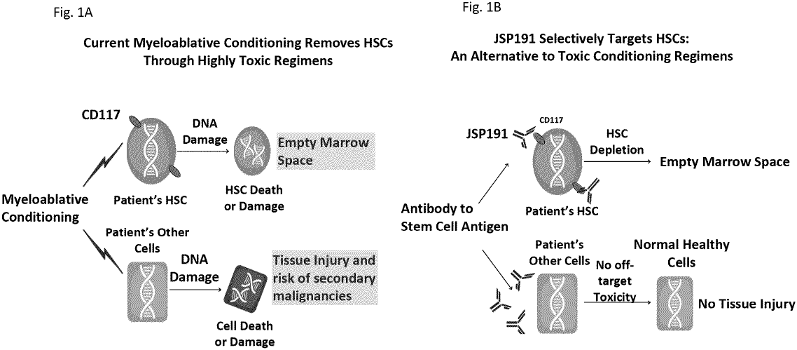

Hematopoietic cell transplant (HCT) can be a curative therapy for many diseases, based on the principle that healthy hematopoietic stem cells (HSCs) and or hematopoietic stem and pluripotent cells (HSPCs) can replace abnormal and diseased HSCs and/or HSPCs. However, HCT is limited in use due to toxicities associated with certain conditioning treatments (FIG. 1A) and low engraftment of donor HSCs/HPSCs. The present disclosure provides non-myeloblative compositions and methods for HCT conditioning that use anti-c-Kit antibodies in combination with total body irradiation (TBI) and chemotherapies (FIG. 1B). These conditioning regimens allow use of lower doses of TBI and result in reduced toxicities, while achieving high levels of donor cell engraftment.

The treatments described herein have been shown to be effective in myelodysplastic syndrome (MDS) and acute myeloid leukemia (AML). However, these treatments are not intended to be limiting to these disease populations alone. Compositions and methods disclosed herein may be used to treat all disorders for which HCT is indicated. Relevant diseases or disorders may include but are not limited to: a cancer, a cardiac disorder, a neural disorder, an autoimmune disease, an immunodeficiency, a metabolic disorder, and/or a genetic disorder, e.g., acute myeloid leukemia, chronic myeloid leukemia, acute lymphoblastic leukemia, hodgkin lymphoma, non-hodgkin lymphoma, neuroblastoma, ewing sarcoma, multiple myeloma, myelodysplastic syndromes, gliomas, thalassemia, sickle cell anemia, aplastic anemia, fanconi anemia, malignant infantile osteopetrosis, mucopolysaccharidosis, pyruvate kinase deficiency, and autoimmune diseases, e.g., multiple sclerosis.

It is to be understood that this invention is not limited to the particular methodology, products, apparatus and factors described, as such methods, apparatus and formulations may, of course, vary. It is also to be understood that the terminology used herein is for the purpose of describing particular embodiments only, and it is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein, “antibody” includes reference to an immunoglobulin molecule immunologically reactive with a particular antigen, and includes both polyclonal and monoclonal antibodies. The term also includes genetically engineered forms such as humanized antibodies, chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Selection of antibodies for endogenous stem cell ablation may be based on a variety of criteria, including selectivity, affinity, cytotoxicity, etc. The phrase “specifically (or selectively) binds” to an antibody, when referring to a protein or peptide, refers to a binding reaction that is determinative of the presence of the protein, in a heterogeneous population of proteins and/or other biologics. Thus, under designated immunoassay conditions, the specified antibodies bind to a particular protein sequence at least two times the background binding and more typically more than 10 to 100 times background binding.

Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into a Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

The selectivity of a particular antibody is typically determined by the ability of one antibody to competitively inhibit binding of the second antibody to the antigen, or by the ability of an antibody to cross-react with multiple epitopes. Any of a number of competitive binding assays can be used to measure competition between two antibodies to the same antigen, or between two antigens to one antibody. An exemplary assay is a BIACORE™ assay. Briefly in these assays, binding sites can be mapped in structural terms by testing the ability of interactants, e.g. different antibodies, to inhibit the binding of another. Injecting two consecutive antibody or antigen samples in sufficient concentration can identify pairs of competing antibodies for the same binding epitope. The antibody samples should have the potential to reach a significant saturation with each injection. The net binding of the second antibody injection is indicative for binding epitope analysis. Two response levels can be used to describe the boundaries of perfect competition versus non-competing binding due to distinct epitopes. The relative amount of binding response of the second antibody injection relative to the binding of identical and distinct binding epitopes determines the degree of epitope overlap.

The term “polynucleotide” refers to a polymeric form of nucleotides of any length, including deoxyribonucleotides or ribonucleotides, or analogs or mixtures thereof. A polynucleotide may comprise modified nucleotides, such as methylated nucleotides and nucleotide or nucleoside analogs, and may be interrupted by non-nucleotide components. If present, modifications to the nucleotide structure may be imparted before or after assembly of the polymer. The term polynucleotide, as used herein, includes, but is not limited to, double- and single-stranded molecules, and mixtures thereof. Unless otherwise specified or required, any embodiment of the invention described herein that is a polynucleotide encompasses both the double-stranded form and each of two complementary single-stranded forms known or predicted to make up the double-stranded form, whether as RNA or DNA, or a mixture thereof.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

A polynucleotide or polypeptide has a certain percent “sequence identity” to another polynucleotide or polypeptide, meaning that, when aligned, that percentage of bases or amino acids are the same when comparing the two sequences. As understood in the art, sequence identity refers to the percentage identity obtained when sequences are aligned for maximum correspondence over a comparison window (e.g., a specified region of each of the sequences), which may be calculated by any of the algorithms described herein using default parameters, which are expected to generate the same alignment, in most cases, when applied to similar sequences. Identity is calculated, unless specified otherwise, across the full length of the reference sequence. Thus, a sequence-of-interest “shares at least x% identity to” a reference sequence if, when the sequence-of-interest is aligned to the reference sequence, at least x% (rounded down) of the residues in the sequence-of-interest are aligned as an exact match to a corresponding residue in the reference sequence. Gaps may be introduced into the sequence-of-interest and/or the reference sequence to maximize correspondence over the comparison window.

Sequence similarity (i.e., identity) can be determined in a number of different manners. To determine sequence identity, sequences can be aligned using the methods and computer programs, including BLAST, available over the worldwide web at ncbi.nlm.nih.gov/BLAST/. Unless indicated to the contrary, sequence identity is determined using the BLAST algorithm (e.g., bl2seq) with default parameters.

Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

The term “native” or “wild-type” as used herein refers to a nucleotide sequence, e.g., gene, or gene product, e.g., RNA or polypeptide, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered before or during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, the term “substantially” means by a significant or large amount or degree. For example, to “substantially” increase may mean to increase by at least two-fold, at least threefold, at least four-fold, at least five-fold, or at least ten-fold, and to “substantially” decrease may mean to decrease by at least 30%, at least 40%, at least 50%, at least 60%, at least 70%, at least 80%, or at least 90%In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988). Conditioning Methods

Prior to hematopoietic cell transplant (HCT), a conditioning therapy may be used for disease eradication, creation of space for engraftment, and/or immunosuppression. In certain embodiments, the disclosure provides methods for conditioning a subject for HCT, the method comprising administering to the subject a combination therapy comprising: a c-Kit inhibitor, total body irradiation (TBI), and a chemotherapeutic agent. In certain embodiments, the method comprises administering to the subject a combination therapy including an anti-c-Kit antibody, total body irradiation (TBI), and a chemotherapeutic agent.

### c-Kit Inhibitors

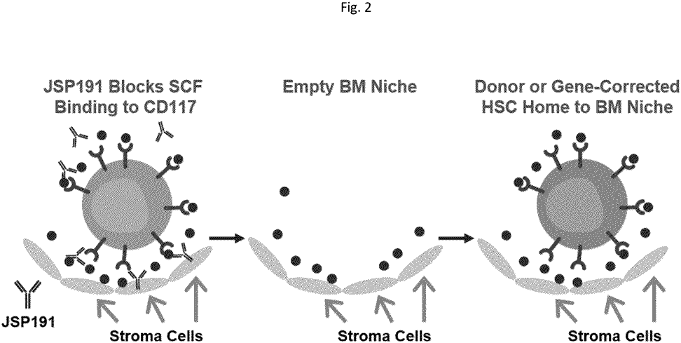

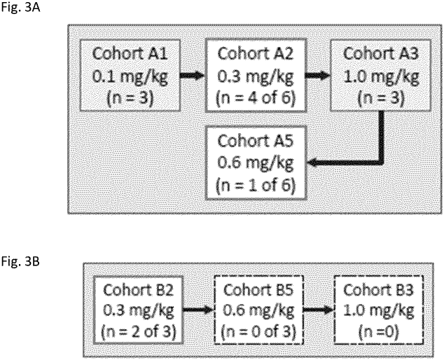

Methods disclosed herein may utilize any inhibitor of c-Kit. The proto-oncogene c-KIT encodes the receptor tyrosine protein kinase KIT, also known as CD117 (a.k.a. cluster of differentiation 117). CD117 binds to stem cell factor (SCF), and also known as mast/stem cell growth factor receptor (SCFR). The interaction of CD117 and SCF is required for stem cell survival. In certain embodiments, c-Kit inhibitors, such as anti-c-Kit antibodies, block SCF from binding to CD117, thereby disrupting critical survival signals, and causing stem cell death. C-Kit inhibitors may therefore function as a conditioning method to clear the bone marrow of endogenous and diseased HSCs and enhance the success of HCT engraftment (FIG. 2).

In certain embodiments, the c-Kit inhibitor inhibits expression of c-Kit, and in certain embodiments, the c-Kit inhibitor inhibits one or more biological activity of c-Kit, such as, e.g., binding to SCF or maintaining stem cell survival. Inhibitors may be any of a variety of molecules, including but not limited to polynucleotides, e.g., single-stranded and/or double-stranded DNA and/or RNA, such as antisense RNA, siRNA, etc., polypeptides, e.g., polypeptides that bind to c-Kit, including but not limited to dominant negative inhibitors and antibodies and antigen-binding fragments thereof, and small molecules (i.e., organic molecules of low molecular weight). Illustrative small molecule inhibitors of c-Kit include but are not limited to: imatinib, dasatinib, pazopanib, quizartinib, sunitinib, midostaurin, etc).

### Anti-c-Kit Antibodies

Compositions and methods disclosed herein may be applicable to any anti-c-Kit antibody, particularly monoclonal anti-human c-Kit antibodies. An anti-c-Kit antibody may refer to an antibody that binds to CD117, e.g., human CD117, or an antigen-binding fragment thereof.

A number of antibodies contemplated by the disclosure that specifically bind human CD117 are known in the art and commercially available, including without limitation JSP-191, SR1, 2B8, ACK2, YB5-B8, 57A5, 104D2, etc. In certain embodiments, the anti-CD117 antibody is selected from the group consisting of: JSP191 (Jasper Therapeutics; Redwood City, CA); CDX-0159 (Celldex Therapeutics, Hampton, NJ); MGTA-117 (AB85) (Magenta Therapeutics, Cambridge, MA); CK6 (Magenta Therapeutics, Cambridge, MA); AB249 (Magenta Therapeutics, Cambridge, MA); and FSI-174 (Gilead, Foster City, CA). Antibodies from Magenta Therapeutics contemplated by the disclosure include but are not limited to those that are disclosed in U.S. Pat. Application Publication No. 20190153114, PCT Application Publication Nos. WO2019084064, WO2020/219748, and WO2020/219770. The FSI-174 antibody is disclosed in PCT application Publication No. WO2020/112687 and U.S. Pat. Application Publication No. 20200165337. The disclosure includes but is not limited to any anti-c-Kit antibodies and/or CDR sets disclosed in any of the patent application disclosed herein, which are all incorporated by reference in their entireties.

In certain embodiments, the anti-c-Kit antibody binds to the extracellular region of CD117, i.e., amino acids 26-524. The sequence of this region is shown below:

QPSVSPGEPSPPSIHPGKSDLIVRVGDEIRLLCTDPGFVKWTFEILDETN
ENKQNEWITEKAEATNTGKYTCTNKHGLSNSIYVFVRDPAKLFLVDRSLY
GKEDNDTLVRCPLTDPEVTNYSLKGCQGKPLPKDLRFIPDPKAGIMIKSV
KRAYHRLCLHCSVDQEGKSVLSEKFILKVRPAFKAVPVVSVSKASYLLRE
GEEFTVTCTIKDVSSSVYSTWKRENSQTKLQEKYNSWHHGDFNYERQATL
TISSARVNDSGVFMCYANNTFGSANVTTTLEVVDKGFINIFPMINTTVFV
NDGENVDLIVEYEAFPKPEHQQWIYMNRTFTDKWEDYPKSENESNIRYVS
ELHLTRLKGTEGGTYTFLVSNSDVNAAIAFNVYVNTKPEILTYDRLVNGM
LQCVAAGFPEPTIDWYFCPGTEQRCSASVLPVDVQTLNSSGPPFGKLVVQ
SSIDSSAFKHNGTVECKAYNDVGKTSAYFNFAFKGNNKEQIHPHTLFTP (SEQ ID NO: 1).

Illustrative anti-c-Kit antibodies include, but are not limited to, SR-1, JSP191, 8D7, K45, 104D2, CK6, YB5.B8, AF-2-1, AF11, AF12, AF112, AF-3, AF-1-1, NF, NF-2-1, NF11, NF12, NF112, NF-3, HF11, HF12, and HF112. A number of antibodies contemplated by the disclosure that specifically bind human CD117 are commercially available, including without limitation SR1, 2B8, ACK2, YB5-B8, 57A5, 104D2, etc. In certain embodiments, the anti-CD117 antibody is selected from the group consisting of: JSP191, CDX-0158 (previously KTN-0158) and CDX-0159 (from Celldex Therapeutics, Hampton, NJ), MGTA-117 (AB85) (from Magenta Therapeutics, Cambridge, MA), CK6 (from Magenta Therapeutics, Cambridge, MA), AB249 (from Magenta Therapeutics, Cambridge, MA), and FSI-174 (from Gilead, South San Francisco, CA). The antibodies from Magenta Therapeutics are disclosed in U.S. Pat. Application Publication No. 20190153114. In certain embodiments, the antibody is one disclosed in any of U.S. Pat. Nos. 7,915,391, US 8,436,150, or US 8,791,249. In certain embodiments, the antibody is one disclosed in U.S. Pat. Application Publ. No. 20200165337 or any of PCT Publication Nos. WO 2020/112687, WO2020/219748, WO 2020/219770, or WO 2019/084064.

In particular embodiments, the antibody is a humanized form of SR1, a murine anti-c-Kit antibody described in U.S. Pat. Nos. 5,919,911 and 5,489,516. The humanized form, JSP191, is disclosed in U.S. Pat. Nos. 7,915,391, 8,436,150, and 8,791,249. JSP191 is an aglycosylated IgG1 humanized antibody. JSP191 specifically binds to human CD117, a receptor for stem cell factor (SCF), which is expressed on the surface of hematopoietic stem and progenitor cells. JSP191 blocks SCF from binding to CD117 and disrupts stem cell factor (SCF) signaling, leading to the depletion of hematopoietic stem cells. JSP191 is a heterotetramer consisting of 2 heavy chains of the IgG1 subclass and 2 light chains of the kappa subclass, which are covalently linked through disulfide bonds. There are no N-linked glycans on JSP191 due to an intentional substitution from an asparagine to glutamine at heavy chain residue 297. The sequences of the heavy chains and light chains of JSP191 are disclosed as SEQ ID NO: 4 from US8436150 and SEQ ID NO: 2 from US8436150, respectively.

The sequences of the heavy chains and light chains of JSP191 are disclosed as SEQ ID NO: 4 from U.S. Pat. No. 8,436,150 and SEQ ID NO: 2 from U.S. Pat. No. 8,436,150, respectively. The sequences of the heavy and light chains of JSP191 are:

### Heavy Chain

MDWTWRVFCLLAVAPGAHSQVQLVQSGAEVKKPGASVKVSCKASGYTFT
SYNMHWVRQAPGQGLEWMGVIYSGNGDTSYNQKFKGRVTITADKSTSTA
YMELSSLRSEDTAVYYCARERDTRFGNWGQGTLVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKF
NWYVDGVEVHNAKTKPREEQYQSTYRVVSVLTVLHQDWLNGKEYKCKVS
NKALPAPIEKTISKAKGQPREPQVYTLPPSRDELTKNQVSLTCLVKGFY
PSDIAVEWESNGQPEN
NYKTTPPVLDSDGSFFLYSKLTVDKSRWQQGNVFSCSVMHEALHNHYTQ
KSLSLSPGK (SEQ ID NO: 2)

and

### Light Chain

MVLQTQVFISLLLWISGAYGDIVMTQSPDSLAVSLGERATINCRASESV
DIYGNSFMHWYQQKPGQPPKLLIYLASNLESGVPDRFSGSGSGTDFTLT
ISSLQAEDVAVYYCQQNNEDPYTFGGGTKVEIKRTVAAPSVFIFPPSDE
QLKSGTASVVCLLNNFYPREAKVQWKVDNALQSGNSQESVTEQDSKDST
YSLSSTLTLSKADYEKHKVYACEVTHQGLSSPVTKSFNRGEC (SEQ ID NO: 3).

In certain embodiments, the variable heavy domain of JSP191 comprises the following sequence:

QVQLVQSGAEVKKPGASVKVSCKASGYTFTSYNMHWVRQAPGQGLEWMGV
IYSGNGDTSYNQKFKGRVTITADKSTSTAYMELSSLRSEDTAVYYCARER
DTRFGNWGQGTLVTVSS (SEQ ID NO: 4).

In certain embodiments, the variable light chain domain of JSP191 comprises the following sequence:

DIVMTQSPDSLAVSLGERATINCRASESVDIYGNSFMHWYQQKPGQPPKL
LIYLASNLESGVPDRFSGSGSGTDFTLTISSLQAEDVAVYYCQQNNEDP YTFGGGTKVEIK (SEQ ID NO:
5).

The CDRs present in JSP191 are as follows: VH CDR1 = YNMH (SEQ ID NO: 6); VH CDR2 = IYSGNGDTSYNQKFKG (SEQ ID NO: 7); VH CDR3 = ERDTRFGN (SEQ ID NO: 8); VL CDR1 = RASESVDIYGNSFMH (SEQ ID NO: 9); VL CDR2 = LASNLES (SEQ ID NO: 10); and VL CDR3 = QQNNEDPYT (SEQ ID NO: 11).

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization. CDX-0159 and other anti-c-Kit antibodies are described in U.S. Pat. No. 10,781,267, and in particular embodiments, an anti-c-Kit disclosed herein comprises the CDRs of any of the antibodies disclosed therein. In certain embodiments, the anti-c-Kit antibody comprises: (i) a light chain variable region (“VL”) comprising the amino acid sequence:

DIVMTQSPSX_{K1}LSASVGDRVTITCKASQNVRTNVAWYQQKPGKAPKX_{K2}LIYS
ASYRYSGVPDRFX_{K3}GSGSGTDFTLTISSLQX_{K4}EDFAX_{K5}YX_{K6}CQQYNSYPRTFG
GGTKVEIK (SEQ ID NO: 12)

, wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain, and X_{K6} is an amino acid with an aromatic side chain; and (ii) a heavy chain variable region (“VH”) comprising the amino acid sequence:

QVQLVQSGAEX_{H1}KKPGASVKX_{H2}SCKASGYTFTDYYINAVVX_{H3}QAPGKGLEWIA
RIYPGSGNTYYNEKFKGRX_{H4}TX_{H5}TAX_{H6}KSTSTAYMX_{H7}LSSLRSEDX_{H8}AVYFCARG
VYYFDYWGQGTTVTVSS (SEQ ID NO: 13)

, wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In specific aspects, described herein are antibodies (e.g., human or humanized antibodies), including antigen-binding fragments thereof, comprising: (i) VH CDRs of a VH domain comprising the amino acid sequence

QVQLKQSGAELVRPGASVKLSCKASGYTFTDYYINWVKQRPGQGLEWIAR
IYPGSGNTYYNEKFKGKATLTAEKSSSTAYMQLSSLTSEDSAVYFCARGV
YYFDYWGQGTTLTVSS (SEQ ID NO: 14) or
QVQLKQSGAELVRPGASVKLSCKASGYTFTDYYINWVKQRPGQGLEWIAR
IYPGSGNTYYNEKFKGKATLTAEKSSSTAYMQLSSLTSEDSAVYFCARGV
YYFDYWGQGTTLTVSA (SEQ ID NO: 15), and

(ii) VL CDRs of a VL domain comprising the amino acid sequence

DIVMTQSQKFMSTSVGDRVSVTCKASQNVRTNVAWYQQKPGQSPKALIYS
ASYRYSGVPDRFTGSGSGTDFTLTI SNVQSEDLADYFCQQYNSYPRTFG
GGTKLEIKR (SEQ ID NO: 16).

MGTA-117 (AB85) is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells, and this antibody and others contemplated by the disclosure are described in U.S. Application Publication No. 20200407440 and/or PCT Application Publication No. WO2019084064. Epitope analysis of AB85 binding to CD177 is described in PCT Application Publication No. WO2020219770, which identified the following two epitopes within CD117:

EKAEATNTGKYTCTNKHGLSNSIYVFVRDPA (SEQ ID NO: 17) (amino acids 60-90), and
RCPLTDPEVTNYSLKGCQGKP (SEQ ID NO: 18) (amino acids 100-130).

The sequences of the variable heavy chain and variable light chains of AB85 are disclosed as SEQ ID NO: 143 and SEQ ID NO: 144 from PCT Application Publication No. WO2019084064, respectively.

The heavy chain variable region (VH) amino acid sequence of Ab85 is:

EVQLVQSGAEVKKPGESLKISCKGSGYSFTNYWIGWVRQMPGKGLEWMAI
INPRDSDTRYRPSFQGQVTISADKSISTAYLQWSSLKASDTAMYYCARHG
RGYEGYEGAFDIWGQGTLVTVSS (SEQ ID NO: 19).

The VH CDR amino acid sequences of AB85 are as follows: NYWIG (VH CDR1; SEQ

ID NO: 20); IINPRDSDTRYRPSFQG (VH CDR2; SEQ ID NO: 21); and HGRGYEGYEGAFDI (VH CDR3; SEQ ID NO: 22).

The light chain variable region (VL) amino acid sequence of AB85 is:

DIQMTQSPSSLSASVGDRVTITCRSSQGIRSDLGWYQQKPGKAPKLLIYD
ASNLETGVPSRFSGSGSGTDFTLTISSLQPEDFATYYCQQANGFPLTFGG
GTKVEIK (SEQ ID NO: 23).

The VL CDR amino acid sequences of AB85 are as follows:

RSSQGIRSDLG (VL CDR1; SEQ ID NO: 24); DASNLET (VL CDR2; SEQ ID NO: 25); and QQANGFPLT (VL CDR3; SEQ ID NO: 26).

FSI-174 is an anti-cKIT antibody being developed in combination with 5F9 as a non-toxic transplant conditioning regimen, as well as a treatment for targeted hematologic malignancies. The sequences of FSI-174 are disclosed in PCT Application Publication No. 2020/112687, U.S. Pat. Application Publication No. 20200165337, and U.S. Pat. No. 11,041,022. In particular embodiments, an anti-c-Kit antibody comprises the three CDRs or variable heavy chain regions present in any of AH1, AH2, AH3, AH4, or AH5 disclosed therein, and/or the three CDRs or variable heavy chain regions present in any of AL1 or AL2 disclosed therein.

In certain embodiments, the CDRs present in FSI-174 and related antibodies are as follows:

VH CDR1 = SYNMH (SEQ ID NO: 27); VH CDR2 = VIYSGNGDTSY(A/N)QKF(K/Q)G (SEQ
ID NO: 28); VH CDR3 = ERDTRFGN (SEQ ID NO: 8); VL CDR1 =
RAS(D/E)SVDIYG(N/Q)SFMH (SEQ ID NO: 29); VL CDR2 = LASNLES (SEQ ID NO: 10);

and

VL CDR3 = QQNNEDPYT (SEQ ID NO: 11).

A/N and the like indicate that the amino acid position may be either of the two amino acids, in this example, A or N. In certain embodiments, CDRs present in the heavy variable region are CDRs H1, H2 and H3 as defined by Kabat: H1 =

SYNMH; H2 = VIYSGNGDTSYAQKFKG (SEQ ID NO: 30); H3 = ERDTRFGN (SEQ ID NO:
8);

and the CDRs present in the light variable region are CDRs L1, L2 and L3 as defined by Kabat:

L1 = RASESVDIYGQSFMH (SEQ ID NO: 31); L2 = LASNLES (SEQ ID NO: 10); and L3 =
QQNNEDPYT (SEQ ID NO: 11)

, respectively except that 1, 2, or 3 CDR residue substitutions is/are present selected from N to A at heavy chain position 60, K to Q at heavy chain position 64 and N to Q at light chain position 30, positions being numbered according to Kabat. In certain embodiments, the antibody comprises any of the heavy chain variable region sequences (AH2, AH3, AH4) and/or light chain variable chain region sequences provided below (AL2), or the CDRs therein shown underlined: AH2:

QVQLVQSGAEVKKPGASVKVSCKASGYTFTSYMNHWVRQAPGQGLEWMGV
IYSGNGDTSYAQKFKGRVTITADKSTSTAYMELSSLRSEDTAVYYCARER
DTRFGNWGQGTLVTVSS (SEQ ID NO: 32)

AH3:

QVQLVQSGAEVKKPGASVKVSCKASGYTFTSYMNHWVRQAPGQGLEWMGV
IYSGNGDTSYNQKFQGRVTITADKSTSTAYMELSSLRSEDTAVYYCARER
DTRFGNWGQGTLVTVSS (SEQ ID NO: 33)

AH4

QVQLVQSGAEVKKPGASVKVSCKASGYTFTSYMNHWVRQAPGQGLEWMGV
IYSGNGDTSYAQKFQGRVTITADKSTSTAYMELSSLRSEDTAVYYCARER
DTRFGNWGQGTLVTVSS (SEQ ID NO: 34)

AL2:

DIVMTOSPLSLPVTPGEPASISCRASESVDIYGOSFMHWYOQKPGOPPKL
LIYLASNLESGVPDRFSGSGSGTDFTLKISRVEAEDVGVYYCOQNNEDPY
TFGGGTKVEIK (SEQ ID NO: 35).

In certain embodiments, the anti-CD 117 antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-CD117 antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-CD117 antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-CD117 antibody comprises a heavy chain or variable region thereof comprising one, two, or three heavy chain CDRs disclosed herein, e.g., a JSP191 heavy chain. In particular embodiments, the anti-CD 117 antibody comprises a light chain or variable region thereof comprising one, two, or three light chain CDRs disclosed herein, e.g., a JSP191 light chain.

In certain embodiments, the anti-c-Kit antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-c-Kit antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-c-Kit antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-c-Kit antibody comprises a heavy chain or variable region thereof comprising one, two, or three heavy chain CDRs disclosed herein, e.g., a JSP191 heavy chain. In particular embodiments, the anti-c-Kit antibody comprises a light chain or variable region thereof comprising one, two, or three light chain CDRs disclosed herein, e.g., a JSP191 light chain.

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization.

MGTA-117 is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells and this antibody and others contemplated by the disclosure are described in US 20200407440.

FSI-174 is an anti-cKIT antibody being develop in combination with 5F9 as a non-toxic transplant conditioning regimen, as well as a treatment for targeted hematologic malignancies.

In particular embodiments, the antibody may include one or more CDR with at least 70%, 80%, 90%, 95%, or 99% amino acid or nucleotide sequence identity to a CDR present in a humanized monoclonal antibody that binds c-Kit, e.g., an antibody derived from any of the mouse antibodies SR1, ACK2, ACK4, 2B8, 3C11, MR-1, and CD122. In some embodiments, the antibody blocks the binding of stem cell factor (SCF) to stem cell factor receptor (CD117). Illustrative embodiments of CD117 antibodies that may be used include JSP191, as well as those described in WO2007127317A2 and US20200165337A1, both incorporated herein in their entirety. In some embodiments, any of the aforementioned antibodies, e.g., JSP191, are administered as part of an HCT conditioning treatment with TBI and a chemotherapy.

### Total Body Irradiation (TBI)

The main purpose of TBI in HSC engraftment conditioning is to suppress the patient’s immune system prior to engraftment. In certain embodiments, the entire patient may be treated with a single radiation beam, with a distance of about 3-6 meters from the radiation source to reduce the dose rate. TBI in extant therapies is typically given in low doses, several times per day, over a period of three to five days. TBI causes significant apoptosis of rapidly dividing cells in radiosensitive organs such as the blood, bone marrow, and the GI tract immediately after radiation exposure. However, in some embodiments, TBI may be given as a single dose as part of a combination conditioning therapy in which an anti-CD117 antibody and a chemotherapy are also administered prior to HSC engraftment. In some embodiments, the TBI is administered at about 100 cGy to about 5 Gy, about 500 cGy to about 5 Gy, about 1 to about 4 Gy, or about 1 to about 3 Gy. In certain embodiments, TBI is administered at about 100 cGy to about 500 cGy, about 200 cGy to about 300 cGy, about 200 cGy, about 250 cGy, or about 300 cGy.

### Chemotherapy

Chemotherapy may refer to any anti-cancer drug that targets rapidly dividing cells. Chemotherapy, i.e., anti-cancer or anti-neoplastic agents may include, but are not limited to, fludarabine, clorafabine, cytarabine, an anthracycline drug, such as daunorubicin (daunomycin) or idarubicin, cladribine (2-CdA), mitoxantrone, etoposide (VP-16), 6-thioguanine (6-TG), hydroxyurea, 6-mercaptopurine (6-MP), azacytidine, and/or decitabine. In certain embodiments, the chemotherapy is fludarabine. In particular embodiments, fludarabine is administered at about 10 mg/m² to about 50 mg/m², about 20 mg/m² to about 40 mg/m², or about 30 mg/m². Chemotherapies may be administered to partially or completely ablate the patient’s bone marrow cells in preparation for donor HSC cell engraftment and/or as part of continuing treatment thereafter. In some embodiments, the chemotherapy, e.g., fludarabine, is administered as part of a combination conditioning therapy in which anti-CD117 antibodies and TBI are also administered prior to HSC engraftment.

The embodiments disclosed herein may be combined and are not intended to be limiting.

### Combinations for Hematopoietic Stem Cell (HSC) Transplant Conditioning

In certain embodiments, the disclosure provides methods for conditioning a subject for HCT, the method comprising administering to the subject an anti-c-Kit antibody, total body irradiation (TBI), and a chemotherapeutic agent. In certain embodiments, the method comprises administering to the subject a JSP191 antibody or variant thereof, TBI, and fludarabine. In certain embodiments, the anti-c-Kit antibody, the total body irradiation (TBI), and the chemotherapeutic agent are administered at the same or different times, or two or more may be administered at the same time, and the other at a different time. In particular embodiments, the anti-c-Kit antibody, the total body irradiation (TBI), and the chemotherapeutic agent are administered to the subject or present within the subject during an overlapping time period prior to the subject receiving HCT.

In some embodiments, the anti-c-Kit antibody is administered about 5 to about 20 days before the HCT. In some embodiments, the anti-c-Kit antibody is administered on days 10 through 14 before the HCT. In some embodiments, the anti-c-Kit antibody is administered on days 5, 6, or 7 through about 10 to about 14 days prior to the HCT. In certain embodiments, the anti-c-Kit antibody is administered daily during any of these time periods. The day of transplant may in some embodiments be determined by the anti-c-Kit antibody blood concentration of the patient: e.g., the day of transplant may occur when the amount of anti-c-Kit antibody remaining in the subject is reduced to a level considered safe for the HCT or at least some transplanted HSC/HSPCs. In particular embodiments, the day of transplant may be within about 4 to about 10 days from the day the subject’s anti-c-Kit antibody blood concentration of about 2000 ng/ml or less.

In some embodiments, the TBI is administered 5, 4, 3, 2, or 1 days prior to the HCT. In other embodiments the TBI is administered the day of the HCT prior to engraftment. In particular embodiments, the TBI is administered once, e.g., on any of the indicated days.

In some embodiments, the chemotherapy is administered on days -10, -9, -8, -6, -7, -5 -4, -3, -2, and/or -1 days prior to the HCT. In certain embodiments, the chemotherapy is administered daily during any of these time periods, or on one or more of these days, e.g., days -2, -3, and -4 relative to the day of HCT.

In some embodiments, the anti-c-Kit antibody (e.g., JSP191 or a humanized c-kit antibody as described in US20200165337A1) is administered on days 14 through 10 prior to HCT, the chemotherapy (e.g., fludarabine) is administered on days 4 through 2 prior to HCT, and the TBI is administered on the day of the transplant, prior to engraftment. In certain embodiments, the antibody and/or chemotherapy is administered daily during any of these time periods. In certain embodiments, the TBI is administered only on a single day.

In some embodiments, the subject is administered about 0.01 mg/kg to about 2 mg/kg of the anti-c-kit antibody, e.g., JSP191, optionally the subject is administered about 0.1 mg/kg to about 1 mg/kg of the anti-c-Kit antibody, e.g., JSP191. In some embodiments, anti-c-Kit antibody may be administered to a subject in a dose about 0.01 mg/kg to about 2 mg/kg of the subject’s body weight, or about 0.1 mg/kg to about 1 mg/kg of the subject’s body weight. In some embodiments, the anti-c-Kit signaling antibodies are administered in a dose of about 0.6 mg/kg, optionally on days 14 through 10 prior to HCT.

In some embodiments, the subject is administered TBI of about 500 cGy to about 5 Gy, optionally of about 1 to about 4 Gy or about 1 to about 3 Gy. In some embodiments, the total body irradiation (TBI) may include a single or fractionated irradiation dose within the range of about 50 cGy - 15 Gy, about 50 cGy - 10 Gy, about 50 cGy - 5 Gy, about 50 cGy - 1 Gy, about 50 cGy -500 cGy, 0.5-1 Gy (500 cGy -1000 cGy), about 0.5-1.5 Gy, about 0.5-2.5 Gy, about 0.5-5 Gy, about 0.5-7.5 Gy, about 0.5-10 Gy, about 0.5-15 Gy, about 1-1.5 Gy, about 1-2 Gy, about 1-2.5 Gy, about 1-3 Gy, about 1-3.5 Gy, about 1-4 Gy, about 1-4.5 Gy, about 1-5.5 Gy, about 1-7.5 Gy, about 1-10 Gy, about 2-3 Gy, about 2-4 Gy, about 2-5 Gy, about 2-6 Gy, or about 2-7 Gy. In some embodiments, the TBI is administered in a single dose of about 2 Gy, optionally within 24 hours prior to the transplant. In some embodiments, the subject is administered twice daily about 2-Gy fractions given over 3 days (total dose about 12 Gy); twice-daily about 1.5-Gy fractions over 4-4.5 days (total dose about 12-13.5 Gy); three-times-daily about 1.2-Gy fractions over 4 days (total dose about 12-13.2 Gy); and once-daily about 3-Gy fractions for 4 days (total dose about 12 Gy). In certain embodiments, a subject is administered low dose TBI, i.e., less than or equal to 5 Gy, e.g., about 1-3 Gy or about 2-4 Gy given in one or two fractions. In particular embodiments, the subject is administered at total of less than about 5 Gy, less than about 4 Gy, less than about 3 Gy, or less than about 2 Gy of TBI, which may be administered in one or more fraction or dose. In particular embodiments, the subject is administered at total of less than about 5 Gy, less than or about 4 Gy, less than or about 3 Hy, less than or about 2 Gy, less than or about 1 Gy, less than about 500 cGy, less than about 250 cGy, less than about 100 cGy, or less than about 50 cGy of TBI, which may be administered in one or more fraction or dose. In particular embodiments, it is administered as a single dose on the day of HCT.

In some embodiments, the subject is administered about 10-50 mg/m²/day of chemotherapy, optionally about 30 mg/m²/day, wherein optionally the chemotherapy is fludarabine and/or clofarabine. In some embodiments, the subject is administered about 10 to about 50 mg/m²/day of the chemotherapy (e.g., fludarabine), optionally 20 mg/m2/day, 25 mg/m²/day, or about 30 mg/m²/day for about one to about six days.

In some embodiments, the subject is administered about 0.1 to about 1.0 mg/kg of the anti-c-Kit antibody (e.g., JSP191 or a humanized c-kit antibody as described in US20200165337A1), about 0.5 to about 3 Gy of the TBI, and about 10-50 mg/m²/day of chemotherapy (e.g., fludarabine), before HCT.

In some embodiments, the anti-c-Kit antibody (e.g., JSP191 or a humanized c-kit antibody as described in US20200165337A1) is administered on days 14 through 10 prior to HCT in a dose of about 0.6 mg/kg, the chemotherapy (e.g., fludarabine) is administered on days 4 through 2 prior to HCT in a dose of about 30 mg/m²/day and the TBI is administered on the day of the transplant, prior to engraftment in a dose of about 2 Gy.

In some embodiments, the anti-c-Kit antibody is administered in a dose of about 0.1 mg/kg to about 1 mg/kg of the anti-c-Kit antibody about 5 to about 20 days before the HCT. In some embodiments, the subject is administered TBI of about 1 to about 3 Gy, about 1-2 days prior to, or on the day of the transplant (day 0). In some embodiments, the subject is administered about 10-50 mg/m²/day of the chemotherapy, optionally about 30 mg/m²/day of the fludarabine and/or clofarabine about 10 to about 1 days prior to the HCT.

In some embodiments, the anti-c-Kit antibody (e.g., JSP191 or a humanized c-kit antibody as described in US20200165337A1) is administered on days -14 through -10 prior to HSC/HSPC transplant in a dose of about 0.6 mg/kg, the chemotherapy (e.g., fludarabine) is administered on three (optionally consecutive) days, e.g., days -4 through -2 prior to HSC/HSPC transplant in a dose of about 30 mg/m²/day, and the TBI is administered on the day of the transplant, optionally prior to transplant or engraftment, in a dose of about 2 Gy. In certain embodiments, the chemotherapy is administered daily during any of these time periods.

In some embodiments, the anti-c-Kit antibody (e.g., JSP191 or a humanized c-kit antibody as described in US20200165337A1) is administered on days 14 through 10 prior to HSC transplant in a dose of about 0.6 mg/kg, the chemotherapy (e.g., fludarabine) is administered on three (optionally consecutive) days, e.g., on days 4 through 2, prior to HSC transplant in a dose of about 30 mg/m²/day, and the TBI is administered on the day of the transplant, optionally prior to transplant or engraftment, in a dose of about 3 Gy. In certain embodiments, the chemotherapy is administered daily during any of these time periods.

In some embodiments, the anti-c-Kit antibody (e.g., JSP191 or a humanized c-kit antibody as described in US20200165337A1) is administered on days 14 through 10 prior to HSC transplant in a dose of about 0.6 mg/kg, the chemotherapy (e.g., fludarabine) is administered on five (optionally consecutive) days, e.g., days 6 through 2, prior to HSC transplant in a dose of about 30 mg/m²/day, and the TBI is administered on the day of the transplant, optionally prior to transplant or engraftment, in a dose of about 2 Gy. In certain embodiments, the chemotherapy is administered daily during any of these time periods.

In some embodiments, the anti-c-Kit antibody (e.g., JSP191 or a humanized c-kit antibody as described in US20200165337A1) is administered on days 14 through 10 prior to HSC transplant in a dose of about 0.6 mg/kg, the chemotherapy (e.g., fludarabine) is administered for five (optionally consecutive) days, e.g., on days 6 through 2, prior to HSC transplant in a dose of about 30 mg/m²/day, and the TBI is administered on the day of the transplant, optionally prior to transplant or engraftment, in a dose of about 3 Gy. In certain embodiments, the chemotherapy is administered daily during any of these time periods.

In some embodiments, the anti-c-Kit antibody is selected from includes one or more, e.g., two, three, four, five, or six, CDRs present in a monoclonal antibody selected from the group consisting of: SR-1, JSP191, 8D7, K45, 104D2, CK6, YB5.B8, AF-2-1, AF11, AF12, AF112, AF-3, AF-1-1, NF, NF-2-1, NF11, NF12, NF112, NF-3, HF11, HF12, and HF112, and is administered on days 14 through 10 prior to HCT in a dose of about 0.6 mg/kg, the chemotherapy (e.g., fludarabine) is administered for 3-5 days (e.g., including day 4 through day 2) prior to HCT in a dose of about 30 mg/m²/day, and the TBI is administered on the day of the transplant, prior to engraftment in a dose of about 2 Gy or about 3 Gy.

In some embodiments, the anti-c-Kit antibody comprises one or more, e.g., two, three, four, five, or six, CDRs present in a humanized version of a monoclonal antibody selected from the group consisting of: ACK2, ACK4, 2B8, 3C11, MR-1, and CD122, and is administered on days 14 through 10 prior to HCT in a dose of about 0.6 mg/kg, the chemotherapy (e.g., fludarabine) is administered for 3-5 days (e.g., including day 4 through day 2) prior to HCT in a dose of about 30 mg/m²/day, and the TBI is administered on the day of the transplant, prior to engraftment in a dose of about 2 Gy or about 3 Gy.

In some embodiments, the anti-c-Kit antibody blocks the binding of stem cell factor (SCF) to stem cell factor receptor (CD117), the antibody (e.g., JSP191) is administered on days 14 through 10 prior to HCT in a dose of about 0.6 mg/kg, the chemotherapy (e.g., fludarabine) is administered for 3-5 days (e.g., including day 4 through day 2) prior to HCT in a dose of about 30 mg/m²/day, and the TBI is administered on the day of the transplant, prior to engraftment in a dose of about 2 Gy or about 3 Gy.

In some embodiments, the anti-c-Kit antibody and/or chemotherapy are present in a pharmaceutical composition. In particular embodiments, the pharmaceutical compositions are in a water-soluble form, such as in pharmaceutically acceptable salts, which is meant to include both acid and base addition salts. Pharmaceutically acceptable acid addition salts include but are not limited to: hydrochloric acid, hydrobromic acid, sulfuric acid, nitric acid, phosphoric acid, organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid. Pharmaceutically acceptable base addition salts include those derived from inorganic bases such as sodium, potassium, lithium, ammonium, calcium, magnesium, iron, zinc, copper, manganese, aluminum salts and the like. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Pharmaceutical compositions as described herein may also include one or more of the following: carrier proteins such as serum albumin; buffers; fillers such as microcrystalline cellulose, lactose, corn and other starches; binding agents; and polyethylene glycol.

The compositions for administration will commonly include an antibody or other ablative agent dissolved in a pharmaceutically acceptable carrier, preferably an aqueous carrier. A variety of aqueous carriers can be used, e.g., buffered saline. The compositions may contain pharmaceutically acceptable auxiliary substances as required to approximate physiological conditions such as pH and buffering agents, toxicity countering agents, e.g., sodium acetate, sodium chloride, potassium chloride, calcium chloride, and sodium lactate. The concentration of active agent in these formulations can vary and are selected based on fluid volumes, viscosities, and body weight in accordance with the particular mode of administration selected and the patient’s needs (e.g., Remington’s Pharmaceutical Science (15th ed., 1980) and Goodman & Gillman, The Pharmacological Basis of Therapeutics (Hardman et al., eds., 1996)).

The anti-c-Kit antibody and/or chemotherapy may be delivered orally, subcutaneously, intravenously, intranasally, transdermally, intraperitoneally, intramuscularly, intrapulmonary, vaginally, rectally, or intraocularly. In some embodiments, the anti-c-Kit antibody, e.g., JSP191, is administered to the subject intravenously, the chemotherapy, e.g., fludarabine, is administered to the subject intravenously, and the TBI is administered in a single dose of radiation.

In certain embodiments, the conditioning regimen is as disclosed in any of the accompanying Examples.

In some embodiments, the subject is also administered one or more of: (a) a graft versus host disease (GVHD) prophylactic agent, optionally selected from the group consisting of glucocorticoids, calcineurin inhibitor, tacromilus, sirolimus, methotrexate, mycophenolate mofetil, mycophenolic acid, cyclosporine A, rapamycin, FK506, corticosteroids, and CD40/CD40L inhibitors; (b) ursodiol; and/or (c) one or more of antibiotic, antifungal, and antiviral therapies.

In some embodiments, the subject is in need of HCT due to a disease or disorder selected from the group consisting of: a cancer, a cardiac disorder, a neural disorder, an autoimmune disease, an immunodeficiency, a metabolic disorder, a bone marrow failure disorder, and a genetic disorder. In some embodiments, the cancer is a solid tissue cancer or a blood cancer, optionally a leukemia, a lymphoma, or a myelodysplastic syndrome (MDS). In particular embodiments, the leukemia is acute myeloid leukemia (AML). In particular embodiments, the lymphoma is diffuse large B-cell lymphoma.

In some embodiments, the disease or disorder is multiple myeloma, severe combined immune deficiency (SCID), chronic myelogenous leukemia (CML), myelodysplastic syndromes (MDS), a myeloproliferative neoplasm, or acute myeloid leukemia (AML).

In certain embodiments, the disease treated according to the disclosure is referred to as MDS/AML. Myelodysplastic syndromes (MDS) and acute myeloid leukemia (AML) exist along a continuous disease spectrum starting with early-stage MDS, which may progress to advanced MDS, AML, cured AML or resistant AML. The disease is characterized by an overproduction of immature blood cells. The resulting lack of mature, healthy blood cells causes anemia and an increased risk for infection and bleeding. Around 5-10% of patients with solid tumors who are treated with chemotherapy, radiation or autologous stem cell transplantation develop treatment-related MDS or AML.

In some embodiments, AML arises from MDS. However, in some embodiments, AML may arise de novo in a subject.

In some embodiments of methods disclosed herein, the subject is a human sixty years or older or 65 years or older. In other embodiments, the subject is an infant and/or is receiving their second HCT. In particular embodiments, the subject is not eligible for myeloablative conditioning. In some embodiments, the subject has a hematopoietic cell transplant comorbidity index (HCT-CI) greater than or equal to 3 (Sorror ML, et al. Hematopoietic cell transplantation (HCT)-specific comorbidity index: a new tool for risk assessment before allogeneic HCT. Blood. 2005;106(8):2912-2919.). In some embodiments, the subject has a hematopoietic cell transplant comorbidity index (HCT-CI) less than or equal to 3. Is particular embodiments, the subject has not previously received a HCT.

### Transplantation Methods

In some embodiments, the disclosure provides methods for HCT, comprising: (i) conditioning a subject in need thereof according to a method disclosed herein; and (ii) transplanting HSCs and/or HPSCs into the subject. In particular embodiments, a hematopoietic cell transplant (HCT) in a mammalian subject includes a method of: (i) conditioning a mammalian subject prior to the HCT with the combination therapy comprising: a CD117 antibody (e.g., JSP191 or variant thereof, or a humanized c-kit antibody as described in US20200165337A1); a chemotherapy (e.g., fludarabine); and TBI; and (ii) transplanting HSCs and/or HPSCs into the subject. In particular embodiments, the transplanted cells are allogenic or autologous to the subject receiving the HCT. In certain embodiments, one or more of the CD117 antibody, chemotherapy, and TBI may be administered prior to, during, or following transplantation of the HSCs and/or HSPCs. In particular embodiments, the conditioning is completed prior to transplantation of the HSCs and/or HSPCs.

In one embodiment, the method comprises:
(i) selectively ablating endogenous hematopoietic stem cells in the subject by administering to the subject an anti-c-Kit antibody (e.g., JSP-191), TBI, and a chemotherapeutic agent (e.g., fludarabine);
(ii) waiting for a period of time following administration of the anti-c-Kit antibody; and
(iii) following (ii), administering to the subject the pharmaceutical composition comprising the HSCs and/or HSPCs, in a dose effective to achieve multilineage peripheral blood chimerism,

In particular embodiments, the period of time of (ii) is sufficient for at least 50%, at least 60%, at least 70%, at least 80%, or at least 90% of the anti-c-Kit antibody to have cleared from the subject’s bloodstream. In certain embodiments, the period of time is between 3 and 20 days. In particular embodiments, the anti-c-Kit antibody, TBI and chemotherapeutic agent are administered at different times, and in certain embodiments, the HCT is performed after administration of the anti-c-Kit antibody, the chemotherapeutic agent, and the TBI.

The term “stem cell” as used herein refers to a mammalian cell that has the ability both to self-renew, and to generate differentiated progeny (see Morrison et al. (1997) Cell 88:287-298). Endogenous stem cells may be characterized by the presence of markers associated with specific epitopes. Hematopoietic stem cells (HSC) are multipotent cells that reside in the bone marrow (BM) and are responsible for the life-long production of mature blood cells. HSPCs include HSCs as well as hematopoietic progenitor cells that reside in bone marrow and are capable of differentiating into mature blood cells.

In some embodiments, HSC and/or HSPC engraftment cells may be fresh, frozen, or subject to prior culture. HSC and/or HSPC may be obtained from fetal liver, bone marrow, cord blood, or peripheral blood, by a donor (allogeneic), the patient themselves (autologous), or any other conventional source.

In some embodiments, HSC and/or HSPC may be genetically altered in order to introduce genes useful in the differentiated cell, e.g., they may comprises a “gene-corrected” repair of a genetic defect in an individual, a selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest or be genetically modified, e.g., to correct a mutation. Methods of gene introduction and gene correction are known in the art, and include, e.g., viral vector-mediated gene delivery, CRISPR, TALEN, and zinc finger-mediated gene correction.

In some embodiments, donor provided unmodified grafts consisting of granulocyte colony-stimulating factor (GCSF)-mobilized peripheral blood stem cells (PBSC) are engrafted into patients. In some embodiments, donors and patients are matched, for example at HLA-A, -B, -C, -DRB1, and -DQB1 by high-resolution typing. In some embodiments, this excludes one HLA class I allele.

For engraftment purposes, a composition comprising hematopoietic stem cells (HSCs) and/or hematopoietic stem and progenitor cells (HSPCs), may be administered to a patient. The HSCs and/or HSPCs are optionally, although not necessarily, purified. Methods are available for purification of stem cells and subsequent engraftment, including flow cytometry; an isolex system (Klein et al. (2001) Bone Marrow Transplant. 28(11):1023-9; Prince et al. (2002) Cytotherapy 4(2):137-45); immunomagnetic separation (Prince et al. (2002) Cytotherapy 4(2):147-55; Handgretinger et al. (2002) Bone Marrow Transplant. 29(9):731-6; Chou et al. (2005) Breast Cancer. 12(3): 178-88); and the like. Each of these references is herein specifically incorporated by reference, particularly with respect to procedures, cell compositions and doses for hematopoietic stem cell transplantation. In particular embodiments, the subject is administered a cell population enriched for CD34+ hematopoietic stem cells, comprising HSCs and/or HSPCs. In some embodiments the cell populations are enriched for expression of CD34, e.g., by art recognized methods such as the cliniMACS.RTM. system, by flow cytometry, etc. Cell populations single enriched for CD34 may be from about 50% up to about 90% CD34+ cells, e.g., at least about 85% CD34+ cells, at least about 90% CD34+ cells, at least about 95% CD34+ cells and may be up to about 99% CD34+ cells or more. Alternatively, unmanipulated bone marrow or mobilized peripheral blood populations are used.

In some embodiments, the HSCs are selected for CD34+ expression, optionally the HSCs are purified, CD34+ Thy-1+ peripheral blood HSCs. In some embodiments, the subject is transplanted with from 10⁵ to 10⁸ CD34+ HSCs/kg of the subject’s body weight, e.g., about 10⁵, about 5x10⁵, about 10⁶, about 5x10⁶, about 10⁷, about 5x10⁷, or about 10⁸ CD34+ HSCs/kg of the subject’s body weight. In some embodiments, the autologous or allogenic HSCs may be gene-corrected.

In some embodiments, the HSCs and/or HSPCs transferred to the subject are autologous to the subject, whereas in other embodiments, they are allogeneic to the subject. In some embodiments, the subject is haploidentical relative to the HSCs. In some embodiments, the HSCs are major histocompatibility complex (MHC) matched to the subject. In some embodiments, subjects receive unmodified grafts consisting of granulocyte colony-stimulating factor (GCSF)-mobilized peripheral blood stem cells (PBSC). In certain embodiments, the HSC/HSPC donor is an HLA matched related or unrelated donor. Donors and recipients may be matched at HLA-A, -B, -C, -DRB 1, and -DQB 1 by high-resolution typing.

In some embodiments, the conditioning and transplantation methods provide for at least 60%, at least 70%, at least 80%, at least 90%, or at least 95% donor CD15 myeloid cell chimerism following the HCT. In some embodiments, the methods provide for full donor chimerism, i.e., at least 95% donor CD15 myeloid cell chimerism following the HCT.

In some embodiments, the conditioning regimen and/or the hematopoietic cell transplant is performed as an outpatient procedure, e.g., wherein the subject arrives at and leaves the clinic the same day as the procedure.

The methods described herein may be used to treat any patient in need of an HCT due to a disease or disorder. The methods disclosed herein may be used to treat a variety of indications amenable to stem cell transplantation. In particular embodiments, HCT methods disclosed herein are used to treat a disease or disorder selected from the group consisting of: a cancer, a cardiac disorder, a neural disorder, an autoimmune disease, an immunodeficiency, a metabolic disorder, hemoglobinopathies, and a genetic disorder. Examples include but are not limited to: acute myeloid leukemia, chronic myeloid leukemia, acute lymphoblastic leukemia, chronic lymphocytic leukemia, hodgkin lymphoma, non-hodgkin lymphoma, neuroblastoma, ewing sarcoma, multiple myeloma, myelodysplastic syndromes, gliomas, thalassemia, sickle cell anemia, aplastic anemia, fanconi anemia, malignant infantile osteopetrosis, mucopolysaccharidosis, pyruvate kinase deficiency, and autoimmune diseases including but not limited to multiple sclerosis or SCIDs.

In some embodiments, the immune disorder is a primary immune deficiency disease, such as but not limited to: severe combined immunodeficiency (SCID), combined immune deficiency (CID), leaky SCID, chronic granulomatous disease (CGD), or common variable immune deficiency (CVID). In certain embodiments, the primary immune deficiency is immunoglobulin G subclass deficiency, selective immunoglobulin A deficiency, DiGeorge syndrome, hyper-immunoglobulin M (HIGM) syndrome, selective IgM deficiency, Wiskott-Aldrich syndrome, or X-linked agammaglobulinemia (XLA).

In some embodiments, the cancer is a solid tissue cancer or a blood cancer, optionally a leukemia, a lymphoma, or a myelodysplastic syndrome (MDS). In some embodiments, the disease or disorder is multiple myeloma, chronic myelogenous leukemia (CML) myelodysplastic syndromes (MDS), a myeloproliferative neoplasm, or a myeloid leukemia, e.g., acute myeloid leukemia (AML) or chronic myeloid leukemia (CML). In some embodiments, the disease is MDS/AML. In some embodiments, the cancer is a lymphoid leukemia, e.g., acute lymphocytic leukemia (ALL) or chronic lymphocytic leukemia (CLL).

In some embodiments, the bone marrow failure disorder is an acquired form or an inherited form of bone marrow failure. Examples of bone marrow failure disorders includes but are not limited to Fanconi anemia (FA), dyskeratosis congenita (DC), Schwachman-Diamond syndrome (SDS), congenital amegakaryocytic thrombocytopenia (CAMT), Blackfan-Diamond anemia (BDA), and reticular dysgenesis (RD).

In particular embodiments, they are used to treat any of the following disorders: multiple myeloma, non-Hodgkin lymphoma, Hodgkin disease, acute myeloid leukemia, neuroblastoma, germ cell tumors, and autoimmune disorders, e.g., systemic lupus erythematosus (SLE), systemic sclerosis, or amyloidosis, for example, by autologous HCT.

In particular embodiments, they are used to treat any of the following disorders: acute myeloid leukemia, acute lymphoblastic leukemia, chronic myeloid leukemia; chronic lymphocytic leukemia, myeloproliferative disorders, myelodysplastic syndromes, multiple myeloma, non-Hodgkin lymphoma, Hodgkin disease, aplastic anemia, pure red cell aplasia, paroxysmal nocturnal hemoglobinuria, Fanconi anemia, thalassemias, thalassemia major, sickle cell anemia, combined immunodeficiency, severe combined immunodeficiency (SCID), Wiskott-Aldrich syndrome, hemophagocytic lymphohistiocytosis (HLH), inborn errors of metabolism (e.g., mucopolysaccharidosis, Gaucher disease, metachromatic leukodystrophies, and adrenoleukodystrophies), epidermolysis bullosa, severe congenital neutropenia, Shwachman-Diamond syndrome, Diamond-Blackfan anemia, leukocyte adhesion deficiency, and the like, for example, by allogeneic HCT.

In some embodiments, the disease is a blood cancer, optionally a leukemia, a lymphoma, or a myelodysplastic syndrome (MDS). In particular embodiments, the methods disclosed are used to treat acute myeloid leukemia (AML), chronic myeloid leukemia (CML), chronic myelomonocytic leukemia (CMML), acute lymphoblastic leukemia (ALL), hodgkin lymphoma, non-hodgkin lymphoma, clonal hematopoiesis of indeterminate potential (CHIP), clonal cytopenia of undetermined significance (CCUS) myelodysplastic syndromes (MDS), idiopathic cytopenia of undetermined significance (ICUS), or myeloproliferative neoplasms (MPN). In particular embodiments, the leukemia is acute myeloid leukemia (AML).

In some embodiments, the disease or disorder is multiple myeloma, chronic myelogenous leukemia (CML) myelodysplastic syndromes (MDS), a myeloproliferative neoplasm, or a myeloid leukemia, e.g., acute myeloid leukemia (AML) or chronic myeloid leukemia (CML). In some embodiments, the disease is MDS or AML. In some embodiments, the cancer is a lymphoid leukemia, e.g., acute lymphocytic leukemia (ALL) or chronic lymphocytic leukemia (CLL).

In some embodiments, the cancer is a myelodysplastic/myeloproliferative neoplasm (MDS/MPN), such as, e.g., chronic myelomonocytic leukemia (CMML). MDS/MPN have both “dysplastic” and “proliferative” features that cannot be classified as either myelodysplastic syndromes (MDS) or myeloproliferative neoplasms (MPN), and for this reason have been categorized as an overlap syndrome with its own distinct characteristics (MDS/MPN). CMML is cancer of the blood. CMML is considered to be one of the myelodysplastic/myeloproliferative neoplasms (MDS/MPN), a type of chronic blood cancer in which a person’s bone marrow does not make blood effectively.

In some embodiments, the subject has a hematopoietic cell transplant comorbidity index (HCT-CI) greater than or equal to 3 (Sorror ML, et al. Hematopoietic cell transplantation (HCT)-specific comorbidity index: a new tool for risk assessment before allogeneic HCT. Blood. 2005;106(8):2912-2919.). In some embodiments, the subject has a hematopoietic cell transplant comorbidity index (HCT-CI) less than or equal to 3.

In some embodiments, the disease or disorder is multiple myeloma, severe combined immune deficiency (SCID), chronic myelogenous leukemia (CML), myelodysplastic syndromes (MDS), a myeloproliferative neoplasm, or acute myeloid leukemia (AML).

In certain embodiments, the disease treated according to the disclosure is referred to as MDS/AML, which includes both MDS and AML. Myelodysplastic syndromes (MDS) and acute myeloid leukemia (AML) exist along a continuous disease spectrum starting with early-stage MDS, which may progress to advanced MDS, AML, cured AML or resistant AML. The disease is characterized by an overproduction of immature blood cells. The resulting lack of mature, healthy blood cells causes anemia and an increased risk for infection and bleeding. Around 5-10% of patients with solid tumors who are treated with chemotherapy, radiation or autologous stem cell transplantation develop treatment-related MDS or AML.

Myelodysplastic syndromes (MDS) are a group of hematopoietic neoplasms characterized by abnormal differentiation and cytomorphology (i.e., dysplasia) of pluripotent hematopoietic progenitor cells (i.e., stem cells) residing in the myeloid compartment of the bone marrow (BM). These abnormalities lead to ineffective hematopoiesis and to cytopenia (i.e., lower-than-normal peripheral blood cell counts) of one or more lineages of the myeloid progenitor cells that manifests as anemia, neutropenia, and/or thrombocytopenia. Methods disclosed herein may be used to treat various forms of MDS, including but not limited to those shown in Table 1 below, which is reproduced from Chung, US Pharm. 2021;46(9):39-44. In certain embodiments, the methods result in decreased cytopenia.

TABLE 1

WHO Classification of MDS
MDS with single-lineage dysplasia (MDS-SLD)
MDS with multilineage dysplasia (MDS-MLD)
MDS with ring sideroblasts (MDS-RS): ≥15% ring sideroblasts in BM or ≥5% with SF3B1 mutation
MDS with ring sideroblasts and single-lineage dysplasia (MDS-RS-SLD)
MDS with ring sideroblasts and multilineage dysplasia (MDS-RS-MLD)
MDS with excess blasts-1 (MDS-EB 1): blasts in blood 2%-4%, blast in BM 5%-9%
MDS with excess blasts-2 (MDS-EB2): blasts in blood 5%-19%, blasts in BM 10%-19%
MDS with isolated 5q-
MDS, unclassifiable (MDS-U)
BM: bone marrow; MDS: myelodysplastic syndromes; WHO: World Health Organization.

In particular embodiments, the methods disclosed are used to treat an immunodeficiency. In particular embodiments, the immunodeficiency is severe combined immunodeficiency (SCID).

In particular embodiments, the methods disclosed are used to treat a genetic disorder. In particular embodiments, the genetic disorder is sickle cell disease or Fanconi anemia. Sickle cell diseases that may be treat include, but are not limited to: HbS disease; drepanocytic anemia; meniscocytosis, and chronic hemolytic anemia.

In some embodiments, the subject has a hematopoietic cell transplant comorbidity index (HCT-CI) greater than or equal to 3 (Sorror ML, et al. Hematopoietic cell transplantation (HCT)-specific comorbidity index: a new tool for risk assessment before allogeneic HCT. Blood. 2005;106(8):2912-2919.).

In some embodiments, the patient is a human sixty years or older or at least 65 years or older. In some embodiments, the patient, e.g., a patient with CML, MDS, or AML, exhibits minimal identifiable disease (MID) and/or measurable residual disease (MRD), which may be detected by techniques including but not limited to cytogenetics, flow cytometry, and/or next-generation sequencing (NGS).

In some embodiments, administration of the combined therapies described herein reduce or rid the patient of MRD. In some embodiments, administration of the combined therapies described herein result in > 50%, > 60%, > 70%, >80%, > 90%, or >95% donor CD15 myeloid chimerism in the peripheral blood of the subject at least 7, 14, or 28 days post HCT. In some embodiments, administration of the combined therapies described herein result in (>95%) donor CD15 myeloid chimerism in the peripheral blood at least 28 days post HCT.

In some embodiments, conditioning using a combination of agents disclosed herein - an anti-c-Kit antibody (e.g., JSP191 or a humanized c-kit antibody as described in US20200165337A1) in a dose of about 0.1 to about 10 mg/kg, optionally about 0.3 mg/kg or 0.6 mg/kg, chemotherapy (e.g., fludarabine), and a low dose of TBI administered prior to engraftment - may be associated with one or more of the following: reduced or no transfusion reaction; reduced or no treatment related toxicities; reduced or no myelosuppression; increased donor HSC engraftment; increased donor myeloid chimerism; and improved clinical outcome. In some embodiments, the subject has not previously had a HCT. In some embodiments, the subject previously received one or more HCT. In some embodiments, the subject is an infant and/or is in need of a second HCT. In some embodiments, the subject is at least 60 or at least 65 years old. In some embodiments, the subject is not eligible for myeloablative conditioning.

In certain embodiments, the disclosure provides a method for treating a subject with a disease or disorder disclosed herein, e.g., AML and/or MDS, optionally wherein the subject is MRD-positive, the method comprising:
(a) conditioning the subject for HCT by administering to the subject:
   (i) an anti-c-Kit antibody, optionally JSP191 or a variant thereof;
   (ii) chemotherapy, e.g., fludarabine; and
   (iii) TBI, optionally at a dose of less than 4 Gy; and then
(b) administering donor HSCs/HSPCs to the subject.

In certain embodiments, the anti-c-Kit antibody is administered first, and the chemotherapy and TBI are administered after the anti-c-Kit antibody is substantially gone from the subject, e.g., substantially cleared from the subject’s blood. In certain embodiments, the subject is administered JSP191 at about 0.6 mg/kg body weight between about Transplant Day (TD)-14 to about TD-10, fludarabine (Flu) at about 30 mg/m²/day for about 3 days (e.g., TD-4, -3, -2), and TBI at 2 Gy on TD0, and then the subject is administered donor HSCs/HSPCs on TD0. In certain embodiments, the subject is administered JSP191 at about 0.6 mg/kg body weight between about Transplant Day (TD)-14 to about TD-10, fludarabine (Flu) at about 30 mg/m2/day for about 3 days (e.g., TD-4, -3, -2), and TBI at 3 about Gy on TD0, and then the subject is administered donor HSCs/HSPCs on TD0, optionally after administration of the TBI.

### EXAMPLES

### Example 1

### Non-Genotoxic Anti-CD117 Transplant Conditioning in Repeat Transplant and Newly Diagnosed Severe Combined Immune Deficiency

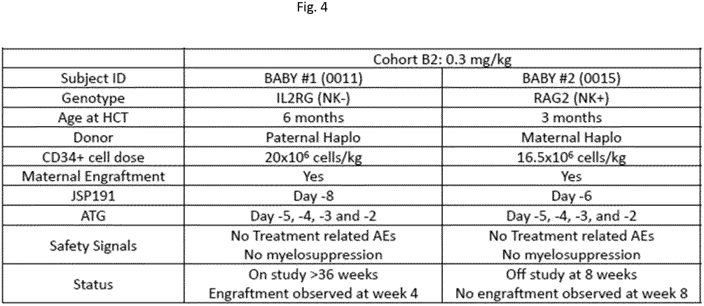

A Phase 1 clinical trial of JSP191 conditioning for HSC engraftment in subjects with severe combined immunodeficiency (SCID), who underwent second transplants because of HSC engraftment failure and poor immunity (PART A) was safe and successful. The study was therefore expanded to include additional cohorts (PART B) of newly diagnosed infants with SCID. Flow charts of dose finding cohorts for patients in re-transplantation and first transplantation infant clinical trials are shown in FIGS. 3A and 3B.

PART A: Clinical study design, SCID re-transplantation population. Specific inclusion criteria included:

SCID defined by Primary Immune Deficiency Treatment Consortium (PIDTC):
Prior donor available
Prior transplant ≥ 6 months
Inadequate B cell engraftment
Incomplete T cell reconstitution
Clinical symptoms due to poor immune function

Status:
No JSP191-related adverse events
Outpatient conditioning
4 of 6 engraftments at 1 year
Open to enrollment

JSP191 was a well-tolerated conditioning regimen. There were no transfusion reactions, nor were there any treatment related toxicities or myelosuppression. PART A subjects were discharged after 48 hours observation following JSP191 administration.

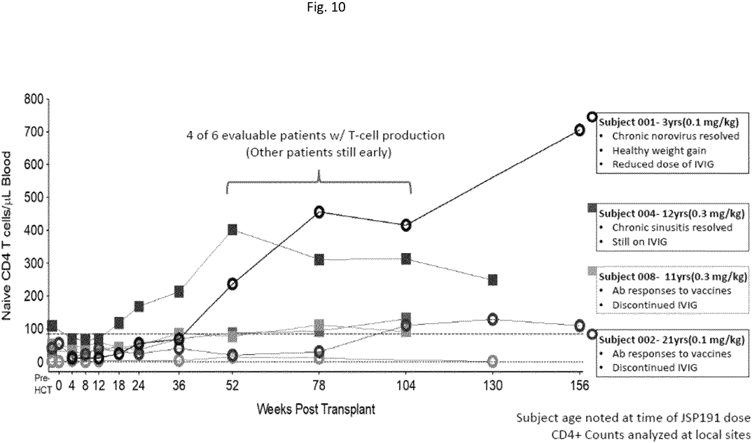

4 out of the 6 patients were able to be evaluated at the time of this application, and all demonstrated CD4+ T-cell production (FIG. 10).

Subject 001 was 3 years old at the time of the JSP191 dose of 0.1 mg/kg. Their chronic norovirus was resolved, healthy weight gain was observed, and their dose of intravenous immunoglobin (IVIG) was reduced.

Subject 004 was 12 years old at the time of the JSP191 dose of 0.3 mg/kg. Their chronic sinusitis was resolved, though they are still on IVIG.

Subject 008 was 11 years old at the time of the JSP191 dose of 0.3 mg/kg. Subject 008 generated antibody responses to vaccines and was able to discontinue IVIG treatment.

Subject 002 was 21 years old at the time of the JSP191 dose of 0.1 mg/kg. Subject 002 also generated antibody responses to vaccines and was able to discontinue IVIG treatment.

Part B: Clinical study design, newly diagnosed SCID first transplant population. FIG. 4 shows the subject demographics of the infant trial. Specific inclusion criteria included:
SCID defined by PIDTC
No prior history of HCT
Haploidentical or HLA matched donor

Status:
No JSP191-related adverse events
1 of 2 infants with engraftment at 9 months
Open to enrollment

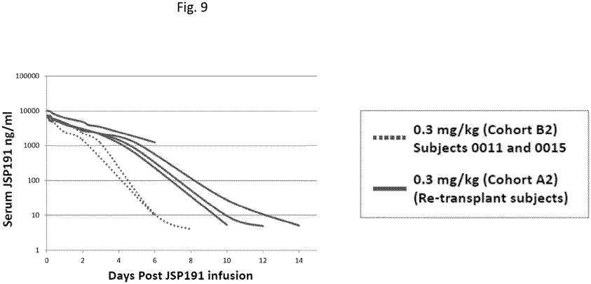

Clearance of JSP191 (FIG. 9) was consistent and predictable in re-transplanted children and adults (ages 11, 12, 12, and 38 years old), and was faster in the newly diagnosed infants (6 and 3 months old).

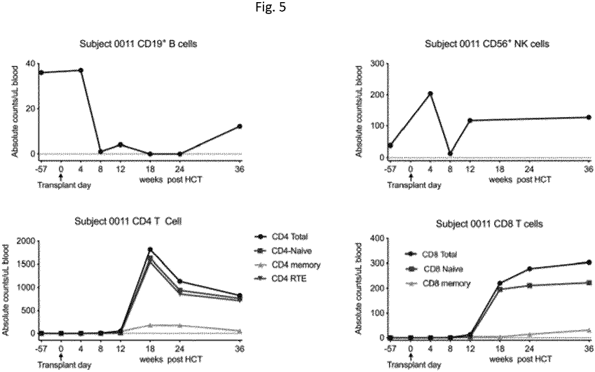

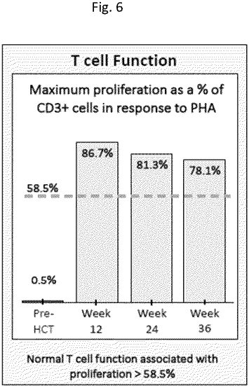

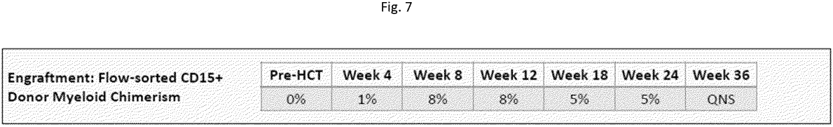

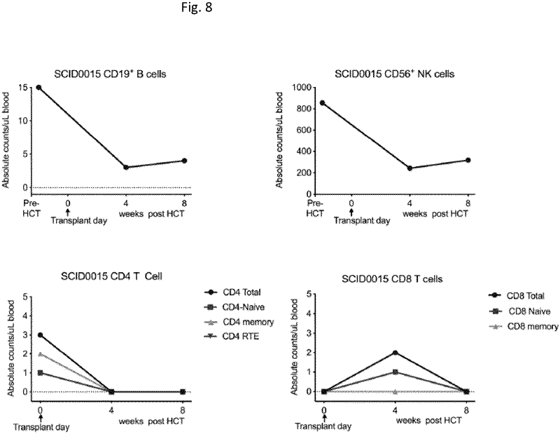

For Part B subjects (newly diagnosed infants), HCT conditioning with JSP191 alone enabled engraftment, immune reconstitution, and transplant function. However the level of CD15+ donor myeloid chimerism was only 5% at week 24. FIGS. 5-7 show T cell and NK cell response in an example infant subject (0011, IL2RG, NK-) in response to hematopoietic cell transplantation (HCT) conditioning with JSP191, the maximum proliferation of T cells, and the percentage of donor myeloid chimerism after engraftment. For Part B subject 0015, FIG. 8 shows T cells and NK cells in this infant subject (0015, RAG2, NK+), in response to HCT conditioning with JSP191 alone. Two SOC tests in subject 0015 exhibited low level maternal engraftment pre-HCT, indicating tolerance of maternal cells. Post-HCT, SOC testing detected only host NK cells in the blood, and all maternal engraftment disappeared, remaining absent through Day + 51. It was clinically determined that the patient had lost tolerance to maternal cells and the patient likely experienced an NK-cell mediated rejection. The clinical decision was then made to proceed with a fully-conditioned HCT from the same donor.

These studies demonstrated that SCID re-transplanted patients, following single agent conditioning with JSP191, achieved durable donor HSC engraftment, chimerism, and clinical benefits (including resolution of chronic infections, independence from IVIG, or antibody response to vaccine challenge). Notably, HSC engraftment in SCID patients is possible without myelosuppression. These studies were the first to demonstrate proof of engraftment of HSC following JSP191 conditioning in an SCID newborn patient, as evidenced by sustained donor myeloid chimerism.

### Example 2

### Phase 1 Study of JSP191, an Anti-CD117 Monoclonal Antibody, With Low Dose Irradiation and Fludarabine in Older Adults with MRD-Positive AML/MDS Undergoing Allogeneic HCT

Myeloablative allogeneic hematopoietic cell transplantation (AHCT) is a potential cure for MDS and AML, but toxicities of conditioning limit its use in older/frail patients. Non-myeloablative (NMA) AHCT is better tolerated but associated with a higher rate of relapse. In this phase 1 study, we evaluated a first-in-class monoclonal antibody (mAb), JSP191, which inhibits stem cell factor binding to CD117 (c-Kit), thereby depleting normal and MDS/AML disease-initiating hematopoietic stem cells (HSC). In pre-clinical models, anti-CD117 mAbs strongly synergize with low dose total body radiation (TBI) to deplete HSC and facilitate donor cell engraftment.

The ability of HCT to achieve successful engraftment following conditioning with JSP191 and other agents was tested. In this Phase 1 clinical trial, we demonstrated that the addition of JSP191 prior to non-myeloablative (NMA) HCT conditioning of 200 cGy total body irradiation (TBI) and fludarabine (Flu) resulted in clearance of disease, lower toxicity, and reduced relapse in older patients with MDS/AML and measurable residual disease (MRD).

Patients with MDS/ AML, > 60 years, with MRD detected by cytogenetics (cyto), difference from normal flow cytometry (flow), or next-generation sequencing (NGS) were eligible for the trial. Specific inclusion criteria included:

Patients with AML or MDS
≥ 60 years or with HCT-CI ≥3
Minimal Identifiable Disease (MID) or Measurable Residual Disease (MRD) detected by cytogenetics (cyto), difference from normal flow cytometry (flow), or next-generation sequencing (NGS)
HLA matched related or unrelated donor
Patients with prior HCT were excluded

Primary endpoints included:
Safety and tolerability of JSP191/TBI/Flu
JSP191 pharmacokinetics (PK)

Secondary endpoints included:
Engraftment and donor chimerism
MRD clearance, Non-relapse mortality, Event-free Survival and Overall Survival
GVHD, NRM, EFS, and OS at 1 year
Depletion of HSPC by JSP191

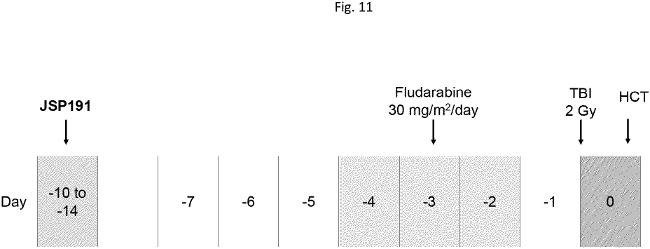

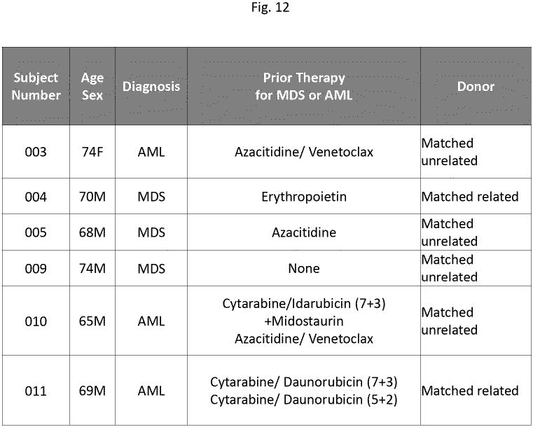

Six eligible subjects were enrolled for the study outlined in FIG. 12. Subject characteristics, prior therapy, and donor information is provided in FIG. 12. JSP191 at 0.6 mg/kg was administered intravenously; serum concentration of JSP191, as determined by PK studies, was used to establish predicted JSP191 clearance and safety to begin Flu at 30 mg/m2/day for 3 days [Transplant Day (TD)-4, -3, -2] and TBI at 2 Gy on TD0, as shown in FIG. 11. Cryopreserved peripheral blood grafts from matched unrelated or matched related donors were administered on TD0, which ranged from 10 to 14 days after JSP191 infusion. GVHD prophylaxis included tacromilus, sirolimus, and mycophenolate mofetil.

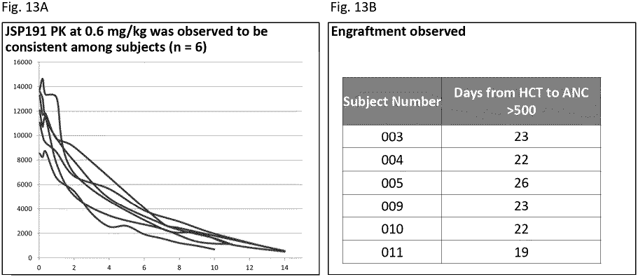

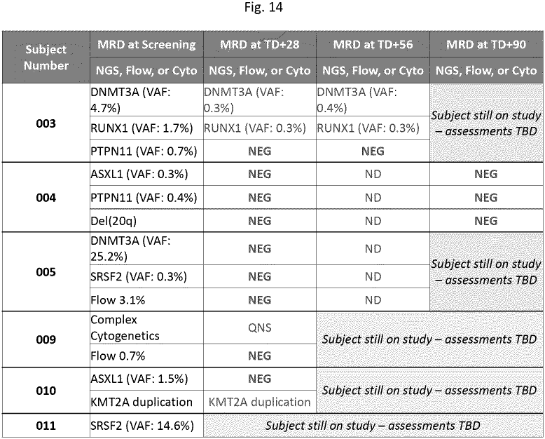

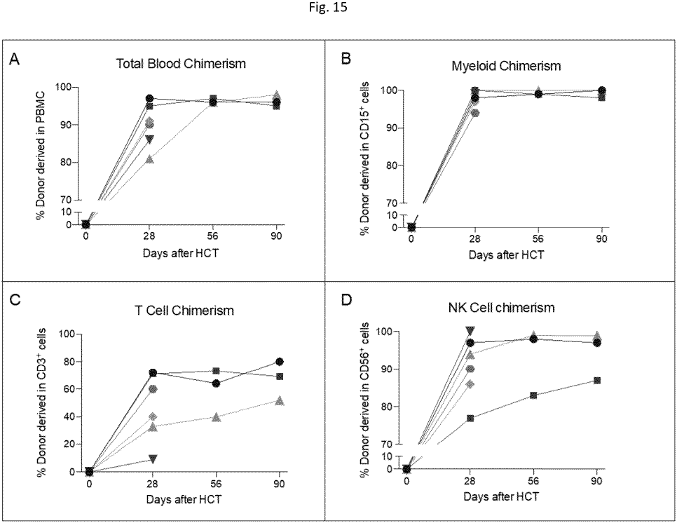

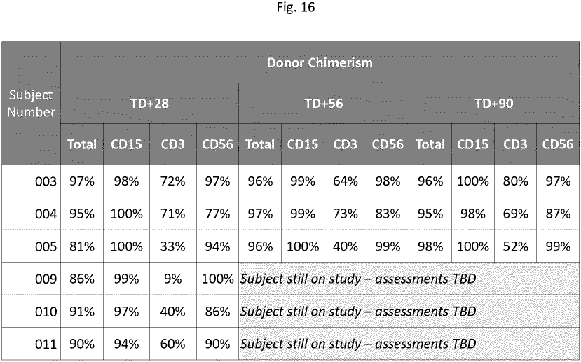

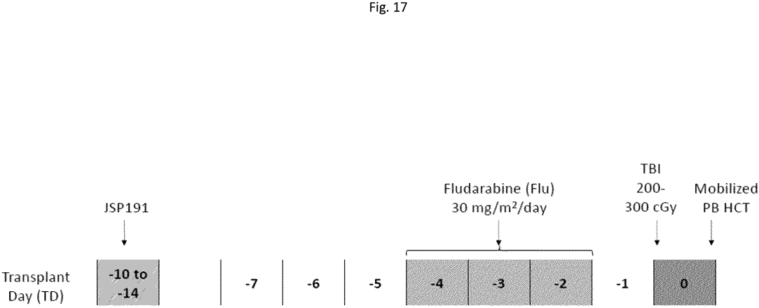

JSP191 PK at 0.6 mg/kg was observed to be consistent among subjects (n=6) as shown in FIG. 13A. All subjects engrafted exhibited neutrophil recovery between TD+19 and TD+26, as shown in FIG. 13B. In contrast, to JSP191 conditioning alone, all subjects receiving the JSP191/TBI/Flu combination therapy, except one, showed evidence of complete (>95%) donor CD15 myeloid chimerism in the peripheral blood at TD+28, and the remaining subject showed evidence of almost complete donor CD15 chimerism (FIG. 15B). All five evaluable subjects with TD+90 follow-up showed complete (>95%) total and myeloid chimerism and MRD elimination (FIG. 16). All subjects showed >80% donor total blood chimerism at TD+28 and beyond (FIG. 15A), and significant CD3 T cell and CD56 NK chimerism at TD+28 and beyond (FIGS. 15C and 15D, FIG. 16). MRD measured at TD+28 showed reduction or elimination of MRD in all subjects tested at TD+28 (FIG. 14 and FIG. 16). There were no infusion toxicities and no JSP191-related serious adverse events.

Following additional subject enrollment, a total of 24 subjects with MDS (n=11) or AML in morphologic CD (n=13) were treated in this study that tested the addition of JSP191 to a standard NMA conditioning of 200-300 cGy TBI and fludarabine (Flu) for safety and eradication of measurable residual disease (MRD) in older adults with high-risk MDS/AML entering AHCT. Total body irradiation (TBI) was increased, after the first 7 subjects for the remaining 17 subjects, to 300 cGy to aid in lymphoablation.

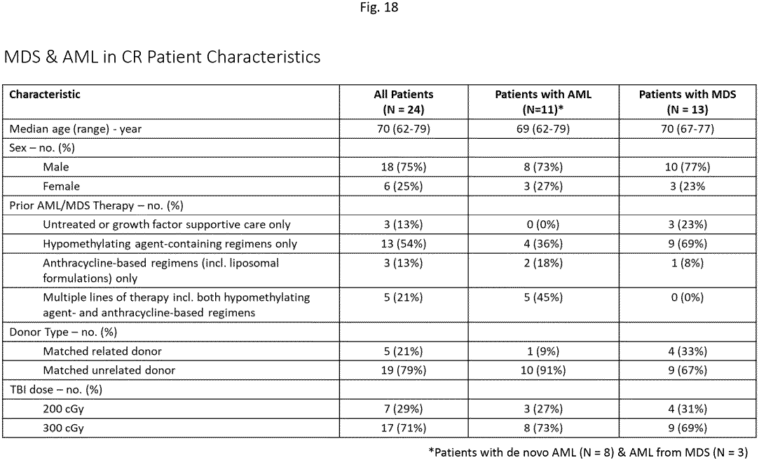

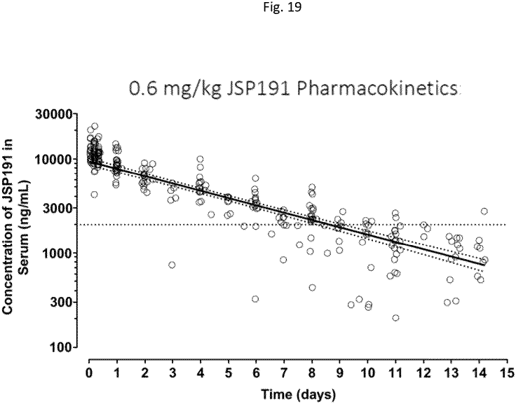

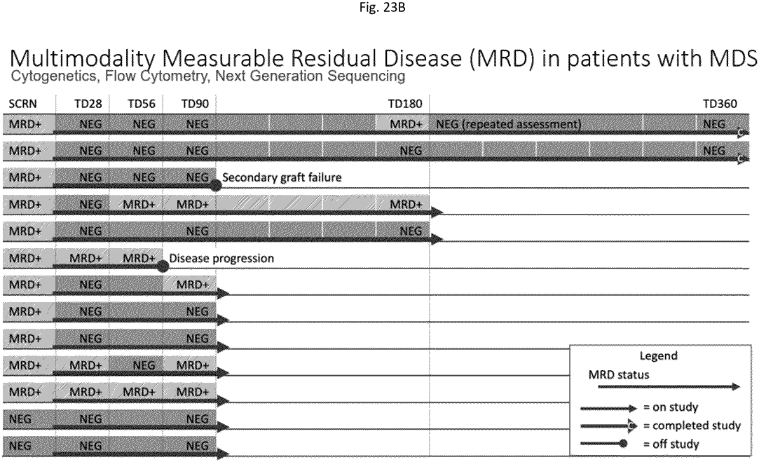

FIG. 18 shows the subject characteristics for the study, including the donor type, prior AML/MDS therapy, and TBI dose. 21% of patients received a transplant from a matched related donor, while 79% of patients received a transplant from a matched unrelated donor. The median age of the subjects was 70 yrs (range 62 to 79). 20 of the 24 subjects had MRD at screening assessed by cytogenetics, flow cytometry, and/or next-generation sequencing (FIGS. 23A and 23B). Pharmacokinetic measurements and modeling of JSP191 were used to determine the Fludarabine (Flu) start date. The pharmacokinetics of 0.6 mg/kg JSP191 in subjects demonstrated consistent and predictable clearance after administration (FIG. 19).

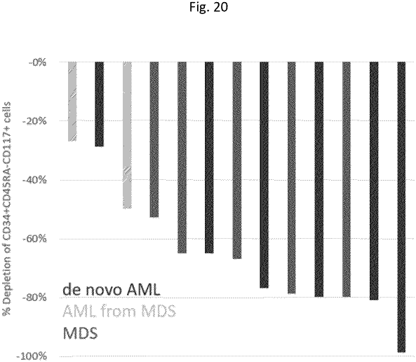

The marrow aspirates of MDS, de novo AML, and AML from MDS subjects were collected from between TD-7 to TD-5 prior to HCT and following JSP191 administration from days TD-10 to TD-14. Notably, the mean percent depletion of HSPC (CD34+CD117+CD45RA-) in the bone marrow of individual subjects 5-7 days after JSP191 alone (prior to administration of Flu/TBI) in the bone marrow of individual MDS and AML subject after receiving JSP191 was 67±25.9% (FIG. 20).

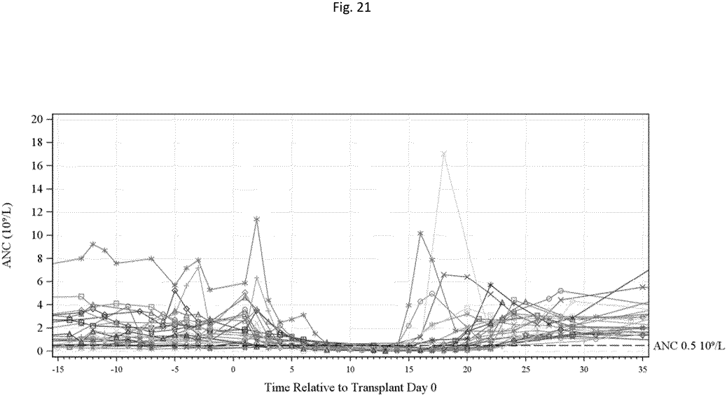

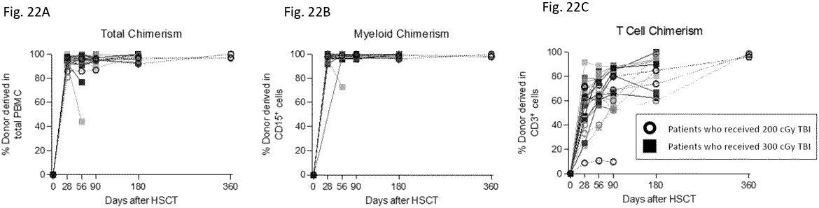

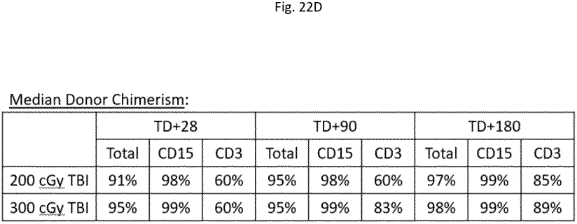

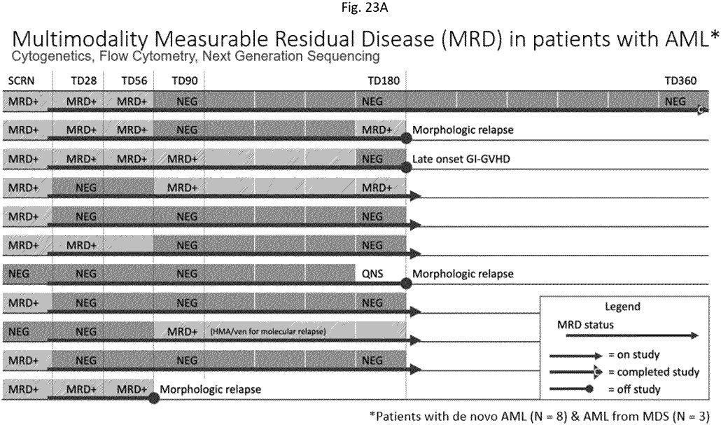

Following JSP191/fludarabine/TBI conditioning and HCT, all subjects displayed neutropenia followed by neutrophil engraftment by TD+26 (FIG. 21). To date, no subjects exhibited infusion toxicities or JSP191-related serious adverse events. All subjects engrafted with neutrophils demonstrated recovery occurring between TD+19 and TD+26. As of TD+180 to TD+360, all the evaluable subjects achieved full myeloid donor chimerism (median 97% total donor chimerism for 200 cGy subjects and median 98% total donor chimerism for 300 cGy subj ects).

Further, MRD clearance was observed in 12 of 20 AML and MDS subjects (FIGS. 23A-23B) to date. No classical grade II-IV acute GVHD has been reported. One case of late onset grade III-IV acute GI GVHD was reported. There has been insufficient follow-up to date to draw conclusions regarding chronic GVHD.

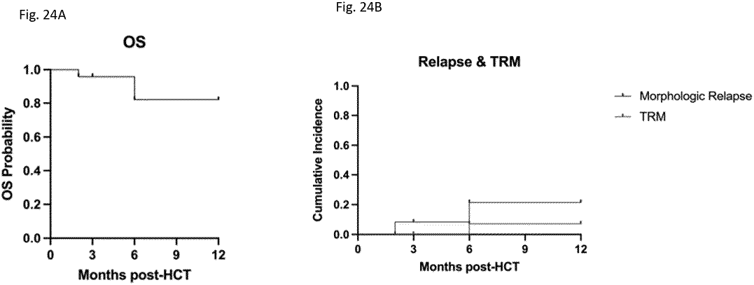

There was a high probability of Overall Survival (OS) (FIG. 24A), and a low incidence of Relapse or Treatment Related Mortality (TRM) (FIG. 24B) 12 months after hematopoietic cell transplant (HCT) with the conditioning regimens provided herein.

### Example 3

### Subanalysis From Phase 1 Study of JSP191, an Anti-CD117 Monoclonal Antibody, in Combination with Low Dose Irradiation and Fludarabine Conditioning, Shows Durable Remissions in Older Adults with Acute Myleoid Leukemia in Complete Remission Undergoing Allogeneic Hematopoietic Cell Transplantation

Allogeneic hematopoietic cell transplantation (HCT) with non-myeloablative conditioning (NMA) is a potential cure for AML in older/frail patients. NMA is associated with better tolerability, but higher relapse rates compared to more intensive regimens. In the phase 1 study of Example 2, JSP191, a monoclonal antibody that inhibits stem cell factor binding to CD117 (c-Kit), was evaluated for its ability to deplete hematopoietic stem and progenitor cells (HSPC), in combination with standard NMA conditioning of low dose total body radiation (TBI) and fludarabine (Flu) for HCT in older adults with AML and MDS. Pre-clinical experiments suggested JSP191 synergizes with low dose TBI to deplete normal and malignant HSPC to facilitate donor cell engraftment. In this Example, a 1 year follow-up of all 12 subjects with AML in morphologic complete remission (CR) is described. MDS subjects were excluded from this subanalysis, because they have significantly shorter median follow up at this time.

Learning Objectives:
1. JSP 191 is an anti-CD 117 antibody that appears to deplete hematopoietic stem and progenitor cells in older AML in CR patients undergoing allogeneic HCT.
2. JSP191 combined Flu/TBI non-myeloablative conditioning regimen appears safe and well-tolerated in older AML in CR patients undergoing allogeneic HCT.
3. JSP191/Flu/TBI facilitates full donor myeloid chimerism and results in clearance of MRD in older AML in CR patients undergoing allogeneic HCT.

### Methods

Twelve subjects, median age 70 yrs (range 62-79), with AML in morphologic CR (CR1 or CR2+) and HLA-matched related or unrelated donors were enrolled in the clinical study described in Example 2. Following infusion of JSP191 0.6 mg/kg, serum levels were assessed to determine when to start Flu at 30 mg/m2/day on Transplant Day (TD) 4, -3, -2, and TBI 2-3 Gy on Transplant Day 0. Peripheral blood grafts were infused on TD0 (10-14 days after JSP191). Graft vs Host Disease (GVHD) prophylaxis administered was tacrolimus, sirolimus, and mycophenolate mofetil. Primary endpoints were safety, tolerability, and JSP191 pharmacokinetics. Secondary endpoints included engraftment, chimerism, MRD clearance, acute Graft vs Host Disease (aGVHD), chronic GVHD (cGVHD) non-relapse mortality (NRM), relapse free survival (RFS), and overall survival (OS) at 1 year.

### Results

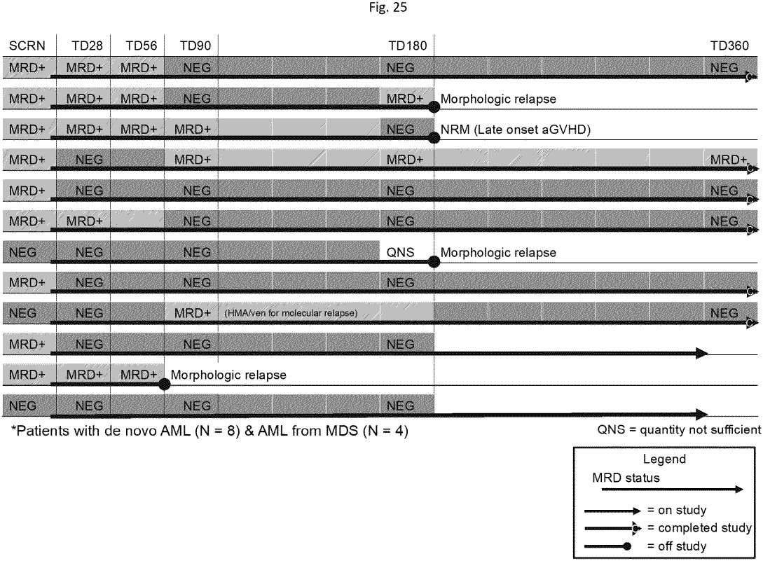

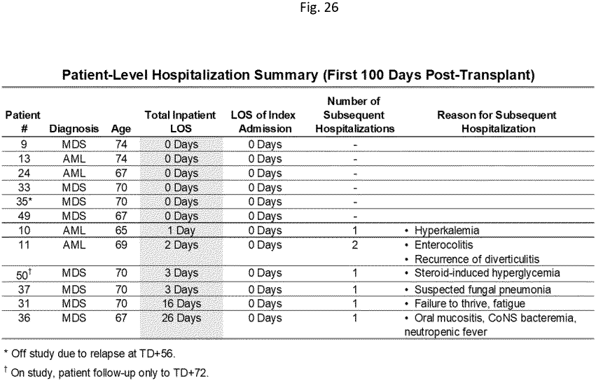

The duration of follow up of each AML subject, minimal residual disease (MRD) status, and outcome at 1 year are summarized in FIG. 25. There were no infusion toxicities and no JSP191-related SAEs. All subjects engrafted, with neutrophil recovery between TD+19 and TD+26. Compared to baseline marrow, JSP191 alone depleted HSPC with a mean decrease of 67.3±25.9% prior to Flu/TBI (FIG. 20). The 11 evaluable subjects at TD+90 achieved full donor myeloid chimerism (mean 98.5±1.3%) and total chimerism of greater than or equal to 94% (mean 95.6±1.3%). Three subjects had grade 2-4 aGVHD, with 1 grade 2 skin aGVHD that resolved, 1 late onset grade 2 skin aGVHD, and 1 NRM due to late onset grade 3 GI aGVHD. Four subjects had moderate cGVHD, and none had severe cGVHD. Three subjects have relapsed, 1 at ~2 months and the others at ~6 months post-HCT. Nine subjects had detectable MRD at pre-HCT screening assessed by cytogenetics, flow cytometry, and/or NGS, 6 of whom were MRD negative at the latest post-HCT evaluable timepoint. Median time to MRD negativity in these subjects was TD+90.

These studies demonstrated that JSP191/TBI/Flu is safe, well-tolerated, and capable of clearing MDS/AML MRD in older adults undergoing non-myeloblative allogeneic HCT (NMA AHCT), thus establishing the combination of an anti-c-Kit antibody, TBI, e.g., low dose TBI, and chemotherapeutic agents, e.g., Flu, as a conditioning regimen suitable for conditioning a variety of patients for HCT. In addition, the combination of JSP191/TBI/Flu resulted in significantly increased donor chimerism as compared to conditioning with JSP191 alone (as shown in Example 1).

### Example 4

### Evaluation of Clinical Outcomes and Healthcare Resource Use of Outpatient Allogeneic Stem Cell Transplant in Older Adults With AML/MDS, Using JSP191, an Anti-CD 117 Monoclonal Antibody, in Combination With Low Dose Irradiation and Fludarabine Conditioning – a Single Center Analysis

Allogeneic stem cell transplant (HCT) patients have a lengthy average inpatient length of stay of 35-45 days in the first 100 days post-HCT, due to the toxicities associated with the preparative conditioning regimens requiring hospitalization (Broder et al, 2017). The clinical outcomes and healthcare resource use of outpatient JSP191 conditioning in combination with low dose irradiation and fludarabine conditioning, as described in Example 2 and Example 3, are provided here. Outpatient HCT enabled by gentler antibody-based conditioning can serve as a strategy to increase available hospital beds and reduce the greater than $250,000 that is spent per patient on HCT in the US, today (Broder et al., 2017; Murthy et al., 2019).

### Methods

The JSP191 phase 1 clinical trial described in Example 2 included patients greater than or equal to 60 years old, with MDS or AML with HLA matched donors, and not eligible for myeloablative conditioning. Clinical outcomes and resource utilization for patients who received outpatient HCT conditioning and donor cell infusion were analyzed. This analysis focuses on the first 100 days post-HCT for 12 subjects who received fully outpatient HCT at a single clinic.

12 subjects with MDS (n=8) or AML in morphologic complete remission (CR, n=4), who received outpatient HCT conditioning and donor cell infusion were treated at a single clinic. The outcomes for these patients are shown in FIG. 28. The median age was 70 years (range 65 to 74), and the median follow-up was 6 months. There were no JSP191 infusion reactions or JSP191-related serious adverse events. All 12 subjects engrafted with neutrophil recovery occurring between TD+15 to TD+26. As of TD+100, we observed 9 infections in 5 patients.

All 12 patients received outpatient the JSP191-based conditioning regimen described in Example 2 and donor cell infusion and were discharged from the hospital the same day, requiring zero inpatient days. 6 of 12 patients did not require an inpatient stay in the first 100 days after transplant. The mean inpatient hospital stay in the first 100 days for all patients was 4 days. Seven total hospitalizations and zero intensive care unit stays were observed.

These results demonstrate that outpatient HCT is clinically feasible and may be associated with lower hospital resource use, while sparing hospitals and patients a long hospitalization.

The various embodiments described above can be combined to provide further embodiments.

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

All of the U.S. patents, U.S. patent application publications, U.S. patent application, foreign patents, foreign patent application and non-patent publications referred to in this specification and/or listed in the Application Data Sheet are incorporated herein by reference, in their entireties.

We claim:

1. A method of conditioning a mammalian subject for a hematopoietic cell transplant (HCT), the method comprising:
(a) administering to the subject an inhibitor of c-Kit, optionally an anti-c-Kit antibody;
(b) administering to the subject total body irradiation (TBI); and
(c) administering to the subject a chemotherapy,
in a dose effective to deplete endogenous hematopoietic stem cells from the subject.

2. The method of claim 1, wherein the anti-c-Kit antibody comprises one or more complementarity-determining regions (CDRs) present in a monoclonal antibody selected from the group consisting of: SR-1, JSP191, MGTA-117, FSI-174, CDX-0159, 8D7, K45, 104D2, CK6, AB249, YB5.B8, AF-2-1, AF11, AF12, AF112, AF-3, AF-1-1, NF, NF-2-1, NF11, NF12, NF112, NF-3, HF11, HF12, and HF112.

3. The method of claim 1, wherein the anti-c-Kit antibody comprises one or more complementarity-determining regions (CDRs) present in a humanized version of a monoclonal antibody selected from the group consisting of: ACK2, ACK4, 2B8, 3C11, MR-1, and CD122.

4. The method of claim 1, wherein the anti-c-Kit antibody comprises the CDRs of an antibody that blocks the binding of stem cell factor (SCF) to stem cell factor receptor (CD117), optionally wherein the antibody is JSP191.

5. The method of any one of claims 1-4, wherein the subject is administered about 0.01 mg/kg to about 2 mg/kg of the anti-c-kit antibody, optionally wherein the subject is administered about 0.1 mg/kg to about 1 mg/kg of the anti-c-Kit antibody.

6. The method of any one of claims 1-5, wherein the subject is administered TBI comprising about 50 cGy to about 5 Gy, optionally wherein the subject is administered TBI comprising about 1 Gy to about 3 Gy.

7. The method of any one of claims 1-6, wherein the subject is administered about 10-50 mg/m²/day of the chemotherapy, optionally wherein the chemotherapy is selected from the group consisting of fludarabine and clofarabine, and optionally wherein the chemotherapy is administered for about one to about six days.

8. The method of any one of claims 1-7, wherein the subject is administered about 0.6 mg/kg of the anti-c-Kit antibody, about 2 Gy of the TBI, and about 30 mg/m²/day of the chemotherapy before the HCT, optionally wherein the anti-c-Kit antibody is JSP191, and optionally wherein the chemotherapy is flutarabine.

9. The method of any one of claims 1-8, wherein the anti-c-Kit antibody is administered to the subject intravenously and/or the chemotherapy is administered to the subject intravenously.

10. The method of any one of claims 1-9, wherein the anti-c-Kit antibody is administered to the subject between about 5 to about 20 days prior to the HCT, optionally between about 10 to about 14 days prior to the HCT.

11. The method of any one of claims 1-10, wherein the level of anti-c-Kit antibody in the subject determines the day of HCT for the subject, optionally wherein the day of transplant is within about 4 to about 10 days from the day the anti-c-Kit antibody is at a concentration of about 2000 ng/ml or less in a subject.

12. The method of any one of claims 1-11, wherein the chemotherapy is administered to the subject between about one to about seven days prior to the HCT, optionally between about two to about four days prior to the HCT, optionally about three days prior to the HCT, and optionally wherein the chemotherapy is administered for about three days.

13. The method of any one of claims 1-12, wherein the TBI is administered to the subject about zero to about three days prior to the HCT, optionally on the same day as the HCT.

14. The method of any one of claims 1-13, wherein the subject is also administered one or more of:
(a) a graft versus host disease (GVHD) prophylactic agent, optionally selected from the group consisting of glucocorticoids, calcineurin inhibitor, tacromilus, sirolimus, methotrexate, mycophenolate mofetil, mycophenolic acid, cyclosporine A, rapamycin, FK506, corticosteroids, and CD40/CD40L inhibitors;
(b) ursodiol; and/or
(c) one or more of antibiotic, antifungal, and antiviral therapies.

15. The method of any one of claims 1-14, wherein the subject is a human sixty years or older.

16. The method of any one of claims 1-15, wherein the subject has a hematopoietic cell transplantation comorbidity index (HCT-CI) greater than or equal to 3.

17. The method of any one of claims 1-16, wherein the subject is in need of a HCT due to a disease or disorder selected from the group consisting of: a cancer, a cardiac disorder, a neural disorder, an autoimmune disease, an immunodeficiency, a metabolic disorder, a bone marrow failure disorder, and a genetic disorder.

18. The method of claim 17, wherein the cancer is a solid tissue cancer or a blood cancer, optionally a leukemia, a lymphoma, or a myelodysplastic syndrome (MDS).

19. The method of claim 18, wherein the cancer is multiple myeloma, acute lymphocytic leukemia (ALL), chronic lymphocytic leukemia (CLL), chronic myelogenous leukemia (CML), myelodysplastic syndromes (MDS), a myeloproliferative neoplasm, or acute myeloid leukemia (AML).

20. The method of claim 17, wherein the immunodeficiency is a primary immune deficiency disease (PIDD), optionally severe combined immunodeficiency (SCID), combined immune deficiency (CID), leaky SCID, chronic granulomatous disease (CGD), or common variable immune deficiency (CVID).

21. The method of claims 17, wherein the bone marrow failure disorder is Fanconi anemia (FA), dyskeratosis congenita (DC), Shwachman-Diamond syndrome (SDS), congenital amegakaryocytic thrombocytopenia (CAMT), Blackfan-Diamond anemia (BDA), or reticular dysgenesis (RD).

22. A method of hematopoietic cell transplant (HCT) in a mammalian subject, the method comprising:
(a) conditioning a mammalian subject according to the method of any one of claims 1-20; and
(b) transplanting hematopoietic stem cells (HSCs) and/or hematopoietic stem and pluripotent cells (HSPCs) into the subject,
wherein step (a) is performed prior to and/or during and/or following step (b), optionally wherein step (a) is completed before step (b).

23. The method of claim 22, wherein the HSCs and/or HSPCs are selected for CD34⁺ expression, optionally wherein the HSCs and/or HSPCs are purified, CD34⁺ Thy-1⁺ peripheral blood HSCs.

24. The method of any one of claims 22-23, wherein the subject is transplanted with from 10⁵ to 10⁸ CD34⁺ HSCs and/or HSPCs /kg of the subject’s body weight.

25. The method of any one of claims 22-24, wherein the HSCs and/or HSPCs are autologous or allogeneic to the subject, optionally wherein the autologous HSCs and/or HSPCs are gene-corrected.

26. The method of any one of claims 22-25, wherein the HSCs and/or HSPCs are derived from bone marrow, cord blood, or peripheral blood of a donor.

27. The method of any one of claims 22-26, wherein the subject is haploidentical relative to the HSCs and/or HSPCs.

28. The method of any one of claims 22-27, wherein the HSCs and/or HSPCs are MHC matched to the subj ect.

29. The method of any one of claims 22-28, wherein the method provides for at least 50%, 60%, 70%, 80%, 90%, or 95% donor CD15 myeloid cell chimerism following the HCT.

30. The method of any one of claims 22-29, wherein minimal residual disease (MRD) and/or measurable residual disease (MRD) is undetected or reduced in the subject after a period of 28 days following the HCT.

31. The method of claim 30, wherein MID and/or MRD are detected by cytogenetics, flow cytometry, and/or next-generation sequencing (NGS).

32. The method of any one of claims 22-29, wherein minimal residual disease (MRD) and/or measurable residual disease (MRD) is undetected or reduced in the subject after a period of 360 days following the HCT.

33. The method of any one of claims 22-32, wherein severe chronic graft versus host disease (cGVHD) is undetected or reduced in the subject after a period of 360 days following the HCT.

34. The method of any one of claims 22-33, wherein step (a) and/or step (b) is performed as an outpatient procedure.


SEQUENCE LISTING (35 sequences)

SEQ ID NO: 1 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 2 - protein, 466 aa, Homo sapiens
MDWTWRVFCLLAVAPGAHSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 238 aa, Homo sapiens
MVLQTQVFISLLLWISGAYGDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Homo sapiens
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 5 - protein, 111 aa, Homo sapiens
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 6 - protein, 4 aa, Homo sapiens
YNMH

SEQ ID NO: 7 - protein, 16 aa, Homo sapiens
IYSGNGDTSYNQKFKG

SEQ ID NO: 8 - protein, 8 aa, Homo sapiens
ERDTRFGN

SEQ ID NO: 9 - protein, 15 aa, Homo sapiens
RASESVDIYGNSFMH

SEQ ID NO: 10 - protein, 7 aa, Homo sapiens
LASNLES

SEQ ID NO: 11 - protein, 9 aa, Homo sapiens
QQNNEDPYT

SEQ ID NO: 12 - protein, 107 aa, synthetic construct
  VARIANT 10: note = X is an amino acid with an aromatic or aliphatic hydroxyl side chain
  VARIANT 46: note = X is an amino acid with an aliphatic or aliphatic hydroxyl side chain
  VARIANT 63: note = X is an amino acid with an aliphatic hydroxyl side chain
  VARIANT 80: note = X is an amino acid with an aliphatic hydroxyl side chain or is P
  VARIANT 85: note = X is an amino acid with a charged or acidic side chain
  VARIANT 87: note = X is an amino acid with an aromatic side chain
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 13 - protein, 117 aa, synthetic construct
  VARIANT 11: note = X is an amino acid with an aliphatic side chain
  VARIANT 20: note = X is an amino acid with an aliphatic side chain
  VARIANT 39: note = X is an amino acid with a polar or basic side chain
  VARIANT 69: note = X is an amino acid with an aliphatic side chain
  VARIANT 71: note = X is an amino acid with an aliphatic side chain
  VARIANT 74: note = X is an amino acid with an acidic side chain
  VARIANT 83: note = X is an amino acid with an acidic or amide derivative side chain
  VARIANT 92: note = X is an amino acid with an aliphatic hydroxyl side chain
QVQLVQSGAEXKKPGASVKXSCKASGYTFTDYYINAVVXQAPGKGLEWIARIYPGSGNTYYNEKFKGRXTXTAXKSTSTAYMXLSSLRSEDXAVYFCARGVYYFDYWGQGTTVTVSS

SEQ ID NO: 14 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSS

SEQ ID NO: 15 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSA

SEQ ID NO: 16 - protein, 108 aa, synthetic construct
DIVMTQSQKFMSTSVGDRVSVTCKASQNVRTNVAWYQQKPGQSPKALIYSASYRYSGVPDRFTGSGSGTDFTLTISNVQSEDLADYFCQQYNSYPRTFGGGTKLEIKR

SEQ ID NO: 17 - protein, 31 aa, Homo sapiens
EKAEATNTGKYTCTNKHGLSNSIYVFVRDPA

SEQ ID NO: 18 - protein, 21 aa, Homo sapiens
RCPLTDPEVTNYSLKGCQGKP

SEQ ID NO: 19 - protein, 123 aa, synthetic construct
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 20 - protein, 5 aa, synthetic construct
NYWIG

SEQ ID NO: 21 - protein, 17 aa, synthetic construct
IINPRDSDTRYRPSFQG

SEQ ID NO: 22 - protein, 14 aa, synthetic construct
HGRGYEGYEGAFDI

SEQ ID NO: 23 - protein, 107 aa, synthetic construct
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 24 - protein, 11 aa, synthetic construct
RSSQGIRSDLG

SEQ ID NO: 25 - protein, 7 aa, synthetic construct
DASNLET

SEQ ID NO: 26 - protein, 9 aa, synthetic construct
QQANGFPLT

SEQ ID NO: 27 - protein, 5 aa, synthetic construct
SYNMH

SEQ ID NO: 28 - protein, 17 aa, synthetic construct
  VARIANT 12: note = A or N
  VARIANT 16: note = K or Q
VIYSGNGDTSYXQKFXG

SEQ ID NO: 29 - protein, 15 aa, synthetic construct
  VARIANT 4: note = D or E
  VARIANT 11: note = N or Q
RASXSVDIYGXSFMH

SEQ ID NO: 30 - protein, 17 aa, synthetic construct
VIYSGNGDTSYAQKFKG

SEQ ID NO: 31 - protein, 15 aa, synthetic construct
RASESVDIYGQSFMH

SEQ ID NO: 32 - protein, 117 aa, synthetic construct
QVQLVQSGAEVKKPGASVKVSCKASGYTFTSYMNHWVRQAPGQGLEWMGVIYSGNGDTSYAQKFKGRVTITADKSTSTAYMELSSLRSEDTAVYYCARERDTRFGNWGQGTLVTVSS

SEQ ID NO: 33 - protein, 117 aa, synthetic construct
QVQLVQSGAEVKKPGASVKVSCKASGYTFTSYMNHWVRQAPGQGLEWMGVIYSGNGDTSYNQKFQGRVTITADKSTSTAYMELSSLRSEDTAVYYCARERDTRFGNWGQGTLVTVSS

SEQ ID NO: 34 - protein, 117 aa, synthetic construct
QVQLVQSGAEVKKPGASVKVSCKASGYTFTSYMNHWVRQAPGQGLEWMGVIYSGNGDTSYAQKFQGRVTITADKSTSTAYMELSSLRSEDTAVYYCARERDTRFGNWGQGTLVTVSS

SEQ ID NO: 35 - protein, 111 aa, synthetic construct
DIVMTQSPLSLPVTPGEPASISCRASESVDIYGQSFMHWYQQKPGQPPKLLIYLASNLESGVPDRFSGSGSGTDFTLKISRVEAEDVGVYYCQQNNEDPYTFGGGTKVEIK